(19) United States
(12) Patent Application Publication
(10) Pub. No.: US 2026/0357197 A1
(43) Pub. Date: Nov. 12, 2026

(54) VASCULARIZED THYROID-ON-A-CHIP AND METHODS OF USE THEREOF

(71) Applicant: The U. S. A., as represented by the Secretary, Department of Health and Human Services., Bethesda, MD (US)

(72) Inventors: Parinaz Fathi, Rockville, MD (US); Anagha Rama Varma, Ellicott City, MD (US); Parnika Kant, Alexandria, VA (US); Nicole Y. Morgan, Bethesda, MD (US); Kaitlyn N. Sadtler, Gaithersburg, MD (US); Madison E. Daminato, Birmingham, MI (US); Paniz Rezvan Sangsari, North Potomac, MD (US)

(73) Assignee: The U. S. A., as represented by the Secretary, Department of Health and Human Services., Bethesda, MD (US)

(21) Appl. No.: 19/247,851

(22) Filed: Jun. 24, 2025

Related U.S. Application Data
(60) Provisional application No. 63/802,302, filed on May 8, 2025.

(51) Int. Cl. C12N 5/071; C12M 1/00; C12M 1/12; C12M 3/00; C12M 3/06

(52) U.S. Cl. CPC C12N 5/0617; C12M 21/08; C12M 23/16; C12M 25/14; C12M 29/00; C12N 2533/90

(57) ABSTRACT

Provided herein are in-vitro models of a thyroid, microfluidic devices, and a thyroid-on-a-chip, and methods of using the foregoing. In particular, the present disclosure provides a novel vascularized in-vitro model of a thyroid having both thyroid follicular cells and thyroid microvascular cells cultured and growing together. Further provided are microfluidic devices having in-vitro vascularized thyroid model embedded therein.

### CROSS-REFERENCE TO RELATED APPLICATION

The present patent application claims the benefit under 35 U.S.C. § 119(e) of U.S. Provisional Patent Application No. 63/802,302, filed on May 8, 2025, the contents of which are incorporated herein by reference.

### STATEMENT REGARDING FEDERALLY SPONSORED RESEARCH OR DEVELOPMENT

This invention was made with Government support under project number 1ZIAEB000102-01 awarded bythe National Institutes of Health, National Institute of Biomedical Imaging and Bioengineering. The Government has certain rights in this invention.

### BACKGROUND OF THE INVENTION

Recently, there has been interest in the development of in-vitro models that can replicate organ function. One organ for which there is significant research interest is the thyroid. The thyroid gland plays a role in metabolism and a variety of processes. However, the thyroid gland is highly vascularized, and, in vivo, thyroid follicular cells and thyroid vasculature cells interact in complex ways. To date, no vascularized thyroid model has been created.

### BRIEF SUMMARY OF THE INVENTION

Provided herein are in-vitro models of a thyroid, microfluidic devices, a thyroid-on-a-chip, and methods of using the foregoing.

In an aspect, the present disclosure provides an in-vitro model of a thyroid, the model comprising: a co-culture comprising two or more thyroid follicular cell (TFC) spheroids or organoids, and thyroid microvasculature endothelial cell (TMEC) microtubules. In embodiments of the in-vitro model of a thyroid, co-culture may be a 3D co-culture. In embodiments, the 3D co-culture is in an extracellular matrix selected from: standard-formulation reconstituted basement membrane extracted from Engelbreth-Holm-Swarm mouse tumor, growth-factor-reduced reconstituted basement membrane extracted from Engelbreth-Holm-Swarm mouse tumor, soluble basement membrane extracted from Engelbreth-Holm-Swarm mouse tumor, and any combination thereof. In embodiments, the two or more TFC spheroids or organoids are derived from TFC-2 cells, TFC-4 cells, or induced pluripotent stem cells (iPSCs). In embodiments, the two or more TFC spheroids or organoids are derived from immortalized human thyroid follicular cells. In embodiments, the immortalized human thyroid follicular cells are HuThyrEC cells. In embodiments, the TMEC microtubules are derived from human primary TMECs or iPSCs. In embodiments, the human primary TMECs are H-6058 cells.

In another aspect, the present disclosure provides a thyroid-on-a-chip comprising a microfluidic device having a chamber defining an enclosed volume, having disposed within the volume the in-vitro model of the present disclosure.

In still another aspect, the present disclosure provides a microfluidic thyroid model device comprising: a microfluidic chamber having disposed therein a co-culture comprising thyroid follicular cells and thyroid microvasculature endothelial cells. In embodiments of the microfluidic thyroid model device, the device comprises a gel disposed within the chamber, wherein at least a portion of the co-culture is disposed within the gel. In embodiments, the gel comprises an extracellular matrix (ECM), wherein the ECM is selected from: standard-formulation reconstituted basement membrane extracted from Engelbreth-Holm-Swarm mouse tumor, growth-factor-reduced reconstituted basement membrane extracted from Engelbreth-Holm-Swarm mouse tumor, soluble basement membrane extracted from Engelbreth-Holm-Swarm mouse tumor, and any combination thereof. In embodiments, the thyroid follicular cells are HuThyrEC cells. In embodiments, the thyroid microvasculature endothelial cells are H-6058 primary human TMECs.

In embodiments of the microfluidic thyroid model device, the microfluidic chamber is teardrop shaped, wherein the teardrop shape defines a wide portion and a narrow tapered portion, and wherein the wide portion of the teardrop-shaped microfluidic chamber is in fluidic communication with a fluid inlet, and the narrow tapered portion of the teardrop-shaped microfluidic chamber is in fluidic communication with a fluid outlet.

Some embodiments of the microfluidic thyroid model device further comprise a pump which causes fluid medium to flow into to fluid inlet, through the chamber, and out the fluid outlet, such that the fluid medium contacts the co-culture as it flows through the chamber. In embodiments, the fluid medium comprises one or more test agents. In embodiments, the one or more test agents is selected from: a thyroid autoimmunity test agent or a thyroid cancer therapeutic agent.

In another aspect, the present disclosure provides a method of testing an agent, the method comprising: (a) contacting the agent with the in-vitro model of the present disclosure, wherein the agent contacting the in-vitro model is suspended in a fluid which is in continuous flow across or through the in-vitro model, and wherein the fluid imparts a shear force on the cells of the in-vitro model; and (b) analyzing the in-vitro model for a response to the agent.

In yet another aspect, the present disclosure provides a thyroid-on-a-chip comprising: a microfluidic device comprising a microfluidic chamber in fluidic communication with a fluid inlet and a fluid outlet; wherein, disposed within the microfluidic chamber, is a vascularized thyroid organoid co-culture comprising two or more thyroid follicular cell (TFC) spheroids or organoids in 3D-culture with thyroid microvasculature endothelial cell (TMEC) microtubules, wherein the TFC spheroids comprise HuThyrEC cells and wherein the TMEC microtubules comprise H-6058 cells, wherein the 3D-culture is in an extracellular matrix selected from: standard-formulation reconstituted basement membrane extracted from Engelbreth-Holm-Swarm mouse tumor, growth-factor-reduced reconstituted basement membrane extracted from Engelbreth-Holm-Swarm mouse tumor, soluble basement membrane extracted from Engelbreth-Holm-Swarm mouse tumor, and any combination thereof. In embodiments, the thyroid-on-a-chip comprises a a vascularized thyroid organoid co-culture comprising two or more TFC spheroids in 3D-culture with TMEC microtubules. In embodiments, the thyroid-on-a-chip comprises a a vascularized thyroid organoid co-culture comprising two or more TFC organoids in 3D-culture with TMEC microtubules.

Additional aspects and embodiments of the invention are as provided in the following detailed description.

### BRIEF DESCRIPTION OF THE THE DRAWINGS

FIG. 1 is an illustrative schematic of a microfluidic device of the present disclosure.

FIG. 2 is a flow diagram of an exemplary protocol for synthesizing an in-vitro model of a thyroid according to the present disclosure.

FIG. 3 is an illustrative model of a microfluidic device having an in-vitro model of a thyroid therein. Inset is a 2-dimensional illustration of a section of a thyroid follicle and thyroid vasculature, showing the thyroid follicle in close proximity to a blood capillary (i.e., thyroid microvasculature).

FIG. 4 is a graphical illustration of an exemplary protocol for synthesizing an in-vitro model of a thyroid according to the present disclosure.

FIG. 5 is a bar graph depicting data illustrating a gene expression profile of HuThyrEC cells versus negative-control Hela cells. Data shown are realtime polymerase chain reaction (RT-PCR) results comparing expression between the HuThyrEC cell line and Hela cells for four key thyroid markers: Thyroid Stimulating Hormone Receptor (TSHR), Thyroglobulin (TG), NKX2-1 (also known as Thyroid Transcription Factor 1 or TTF-1), and PAX8.

FIGS. 6A-6F are a series of fluoresce microscopy images of key thyroid markers in Hy ThyrEC cells. For each of FIGS. 6A-6F, the first column labeled “Nucleus” depicts micrographs of a first fluorescence channel; the second column, labeled “Actin”, depicts micrographs of a second fluorescence channel; the third column, labeled according to the immunostained protein, depicts micrographs of a third fluorescence channel; and the fourth column depicts micrographs of the three channels merged together. For each of FIGS. 6A-6F, the upper rows, labeled “Secondary Only” show negative controls that were not incubated with primary antibody; the lower rows, labeled “With Primary,” show the primary-antibody-stained samples. FIG. 6A shows immunofluorescence staining results for the thyroid marker NIS (Sodium/Iodide Symporter, also known as SLC5A5). As seen in the micrograph in row 2, column 3, NIS appears all around the cell when primary antibody is present. FIG. 6B shows immunofluorescence staining results for the thyroid marker NKX2-1. NKX2-1 is a transcription factor, and therefore, as seen in the micrograph in row 2, column 3, localizes in the cell nuclei. FIG. 6C shows immunofluorescence staining results for the thyroid marker PAX8. PAX8 is a transcription factor, and therefore, as seen in the micrograph in row 2, column 3, localizes in the cell nuclei. FIG. 6D shows immunofluorescence staining results for the proliferation marker Ki-67 (also known as MKI67). As seen in the micrograph in row 2, column 3, Ki-67 appears in pockets around the cell when primary antibody is present. FIG. 6E shows immunofluorescence staining results for the cell-cell adhesion marker ZO-1 (also known as TJP1). ZO-1 is a cell adhesion marker, and therefore, as seen in the micrograph in row 2, column 3, localizes at the surfaces of the cells. And FIG. 6F shows immunofluorescence staining results for the thyroid marker TSHR. As seen in the micrograph in row 2, column 3, TSHR appears throughout the cells when primary antibody is present. For each image shown in FIGS. 6A-6F, the scale is 300 μm across.

FIG. 7 is a fluorescence microscopy comparison of ZO-1 expression in HuThyrEC cells versus Caco-2 human colon adenocarcinoma intestinal epithelium cells (positive control for ZO-1). The the first column, labeled “Nucleus,” depicts micrographs of a first fluorescence channel; the second column, labeled “Actin,” depicts micrographs of a second fluorescence channel; the third column, labeled “ZO-1,” depicts micrographs of a third fluorescence channel; and the fourth column depicts micrographs of the three channels merged together. The top two rows show immunofluorescence micrographs for the positive control Caco-2 cells. The bottom two rows show immunofluorescence micrographs for HuThyrEC cells of the present disclosure. For each image shown in FIG. 7, the scale bar is 75 μm.

FIG. 8 is a time series of nine micrographs of a well plate well showing a growth series of thyroid spheroids over ten days. For each image shown in FIG. 8, the scale bar is 750 μm.

FIG. 9 a graph depicting data points illustrating thyroid spheroid growth (in diameter, μm) over ten days of the thyroid spheroid imaged in FIG. 8.

FIGS. 10A-10B are time series images showing live/dead immunofluorescence staining of spheroids across over nine days. For each of FIG. 10A and FIG. 10B, the top row shows a first fluorescence channel, which is stained for live cells, and the bottom row shows a second fluorescence change, which is stained for dead cells. FIG. 10A shows live/dead immunofluorescence staining for days 2, 3, and 4 of thyroid spheroid growth. FIG. 10B shows live/dead immunofluorescence staining for days 6, 8, and 9 of thyroid spheroid growth. For each image shown in FIGS. 10A-10B, the scale is 600 μm across.

FIG. 11 is a series of three confocal immunofluorescence micrographs showing cell proliferation, taken at day 4 of thyroid spheroid growth. The first image shows the cell nuclei in a first fluorescence channel, the middle image shows cell edges in a second fluorescence channel, and the right image shows the two challenges merged together. For each image shown in FIG. 11, the scale bar is 200 μm.

FIG. 12 is a series of micrographs showing spheroids grown under various starting conditions, including HuThyrEC-2 variant cells vs. HuThyrEC-4 variant cells; starting plating density; and regular growth medium vs. hydroxypropyl methylcellulose (HPMC) medium.

FIGS. 13A-13B show HuThycEC-4 thyroid spheroids grown in 384-well growth plates. FIG. 13A shows the thyroid spheroids after three days of growth. FIG. 13B shows the thyroid spheroids after five days of growth. In each of FIGS. 13A-13B, the top row depicts spheroids of the lowest seeding cell density, and each successive row shows spheroids of increasing seeding cell density. The bottom row depicts spheroids of the highest seeding cell density. Increased seeding density resulted in larger spheroids, as did longer time in culture.

FIGS. 14A-14B show HuThycEC-2 organoids (FIG. 14A) and HuThycEC-4 organoids (FIG. 14B) grown at various starting seeding densities, which were seeded in a standard-formulation reconstituted basement membrane 3D-culture medium extracted from Engelbreth-Holm-Swarm (EHS) mouse tumor. Lower seeding densities and less time in culture yielded organoids with less network formation.

FIG. 15 shows a time series of thyroid organoid growth at three different magnifications at two days, four days, seven days, and nine days of growth. Scale for each row is indicated in the first column.

FIGS. 16A-16B are line graphs depicting data illustrating thyroid organoid characteristics from two days to ten days of growth. FIG. 16A is a graph showing organoid diameter (um) over time measured from image analysis. FIG. 16B is a graph showing organoid circularity over time measured from image analysis.

FIG. 17 is a bar graph showing fold change in gene expression of four thyroid cell markers in thyroid organoids after ten days of growth. For each of the four gene markers shown, the left bar relates to a negative control and the right bar relates to the tested organoid.

FIG. 18 is a bar graph showing fold change in gene expression of four thyroid cell markers in thyroid organoids grown in 3D culture versus thyroid cells grown on 2D culture plates. As shown in FIG. 18, for each of the four genes on the x-axis, the bar on the left refers to cells, and the bar on the right refers to organoids.

FIG. 19 is a bar graph showing fold change in gene expression of four thyroid cell markers in thyroid organoids where the growth medium was changed every day (left bar for each test gene) versus where the growth medium was changed every other day (right bar for each test gene).

FIG. 20 is a bar graph showing fold change in gene expression of four thyroid cell markers in thyroid organoids taken at Day 2 time point, Day 4 time point, and Day 9 time point. For each time point, the leftmost bar relates to TSHR, the second bar relates to TG, the third bar relates to NKX2-1, and the fourth bar relates to PAX8.

FIGS. 21A-21B are immunofluorescence micrographs of thyroid organoids imaged on inverted fluorescence microscope. For each of FIGS. 21A-21B, the top row shows negative control incubated with secondary antibody only, and the bottom row shows test sample incubated with antibody stain. The left columns show a first fluorescence channel, and middle columns show a second fluorescence channel, and the right columns show the merged images. In FIG. 21A, the thyroids organoids were tested for cell adhesion marker ZO-1. In FIG. 21B, the thyroid organoids were tested for cell proliferation marker Ki-67. Scale bars are indicated in the images.

FIG. 22 depicts immunofluorescence micrographs of a two thyroid organoids imaged on confocal microscope, the first organoid shown in the top row and the second organoid shown in the bottom row. The left column shows a first fluorescence channel, with the cell nuclei stained; the right column shows a second fluorescence channel with actin stained.

FIGS. 23A-23B depict images of a growth time course of thyroid organoids grown in various culture media and co-culture conditions. FIG. 23A shows thyroid organoids grown in Matrigel®. The left column shows hy ThyrEC-2-only monoculture organoids. The second column shows hy ThyrEC-2 organoids co-cultured with human thyroid microvasculature endothelial cells (HTMECs). The middle column shows HTMECs monocultures seeded on their own without thyroid organoid. The fourth column shows huThyrEC-4 organoids co-cultured with HTMECs. The fifth column shows huThyrEC-4-only monoculture organoids. FIG. 23B shows thyroid organoids grown in Cultrex™ Basement Membrane Extract (BME). The left column shows hy ThyrEC-2-only monoculture organoids. The second column shows hy ThyrEC-2 organoids co-cultured with human thyroid microvasculature endothelial cells (HTMECs). The middle column shows HTMECs monoculture seeded on their own without thyroid organoid. The fourth column shows huThyrEC-4 organoids co-cultured with HTMECs. The fifth column shows huThyrEC-4-only monoculture organoids.

FIG. 24 depicts images of time courses, imaged at Day 1 time point, Day 2 time point, Day 4 time point, and Day 8 time point, of huThyrEC-4 thyroid organoids grown in three different 3D growth media. The left column shows huThyrEC-4 thyroid organoid grown in Matrigel®; the middle column shows huThyrEC-4 thyroid organoid grown in growth-factor-reduced (GFR) Matrigel®; and the right column shows huThyrEC-4 thyroid organoid grown in Cultrex™ BME.

FIGS. 25A-25B are images depicting monocultures of HTMECs grown in different 3D culture media, taken 24 hours after seeding (FIGS. 25A) and 48 hours after seeding (FIG. 25B). As used in FIGS. 25A-25B, “TSH” refers to thyroid stimulating hormone. The top row depicts HTMEC monocultures grown without TSH. The second row depicts HTMEC monocultures grown in 0.1 μM TSH. The third row depicts HTMEC monocultures grown in 0.5 μM TSH. The bottom row depicts HTMEC monocultures grown in 1 μM TSH. In each of FIGS. 25A-25B, left column shows HTMEC monocultures grown in Matrigel®; the middle column shows HTMEC monocultures grown in growth-factor-reduced (GFR) Matrigel®; and the right column shows HTMEC monocultures grown in Cultrex™ BME.

FIGS. 26A-26B are two graphs showing growth data of thyroid organoids grown in varying 3D culture media. FIG. 26A is a bar graph showing mean diameter of organoids after ten days of growth in each of Matrigel®, GFR Matrigel®; and Cultrex™ BME. FIG. 26B is a histogram of all the measured organoids after ten days growth in each of Matrigel®, GFR Matrigel®; and Cultrex™ BME.

FIGS. 27A-27D are bar graphs depicting relative gene expression levels for thyroid organoids grown in three different 3D culture growth media of four thyroid expression markers.

FIGS. 28A-28C are series of images showing thyroid organoid monocultures (left column of each of FIGS. 28A-28C) and thyroid organoid+HTMEC co-cultures (right column of each of FIGS. 28A-28C). FIG. 28A shows thyroid organoid monocultures and thyroid organoid+HTMEC co-cultures grown in Matrigel® medium at four different concentration levels of TSH. FIG. 28B shows thyroid organoid monocultures and thyroid organoid+HTMEC co-cultures grown in GFR-Matrigel® medium at four different concentration levels of TSH. FIG. 28C shows thyroid organoid monocultures and thyroid organoid+HTMEC co-cultures grown in Cultrex™ BME medium at four different concentration levels of TSH.

FIGS. 29A-29C are various views of 3D computer model simulations of fluid flow in and through the chamber of a microfluidic tear-drop shaped chamber having a fluid inlet on the wide portion of the “teardrop” and fluit outlet at the narrow portion of the “teardrop.” FIG. 29A shows a perspective view of a 3D computer model, modeling fluid flow streamlines. FIG. 29B shows an overhead view of a 3D computer model, modeling fluid velocity of fluid pumped into and through the chamber. FIG. 29C shows a perspective view of a 3D computer model, modeling fluid pressure of fluid pumped into and through the chamber.

FIGS. 30A-30C are images taken of thyroid follicular cells inside the chamber of a microfluidic device and with fluid flow active. FIG. 30A shows thyroid follicular cells on gel, stained with green fluorescent membrane-bound dye. FIG. 30B shows thyroid microscular cells on gel. FIG. 30C is a still image taken from live video of thyroid follicular cells in a microfluidic device with fluid flow active.

### DETAILED DESCRIPTION OF THE INVENTION

The mammalian thyroid is a butterfly-shaped organ that is responsible for the production of thyroxine and triiodothyronine, which are hormones involved in the regulation of processes ranging from organ function to digestion. Disorders of the thyroid, such as autoimmune diseases and cancers, are complex conditions that can interrupt many of these processes.

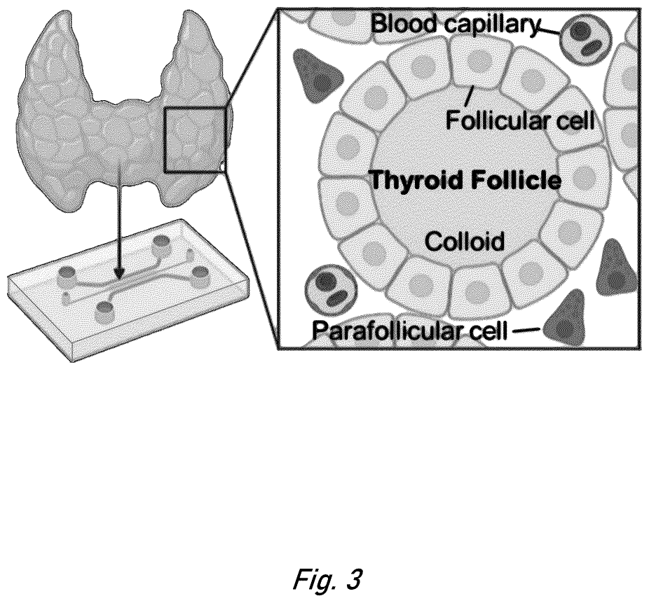

The thyroid produces hormones that play a role in metabolism and development. The thyroid gland is highly vascularized and is primarily composed of spherical follicles formed by thyroid follicular cells (TFCs). (See FIG. 3 inset.)

Microphysiological models of the thyroid can provide a platform through which the thyroid immune microenvironment, thyroid toxins, and thyroid cancers can be studied, and potential therapeutics can be evaluated.

In realistic physiological conditions, cells in thyroid follicles and cells in the intricate, dense network with microvasulature release biomolecular signals that cause TFCs and thyroid microvasculature endothelial cells (TMECs) to mutually interact.

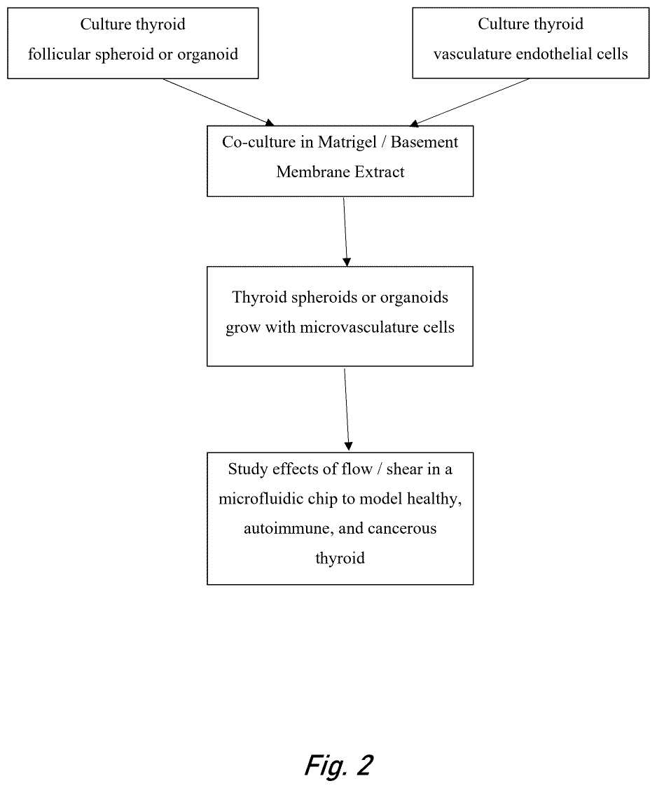

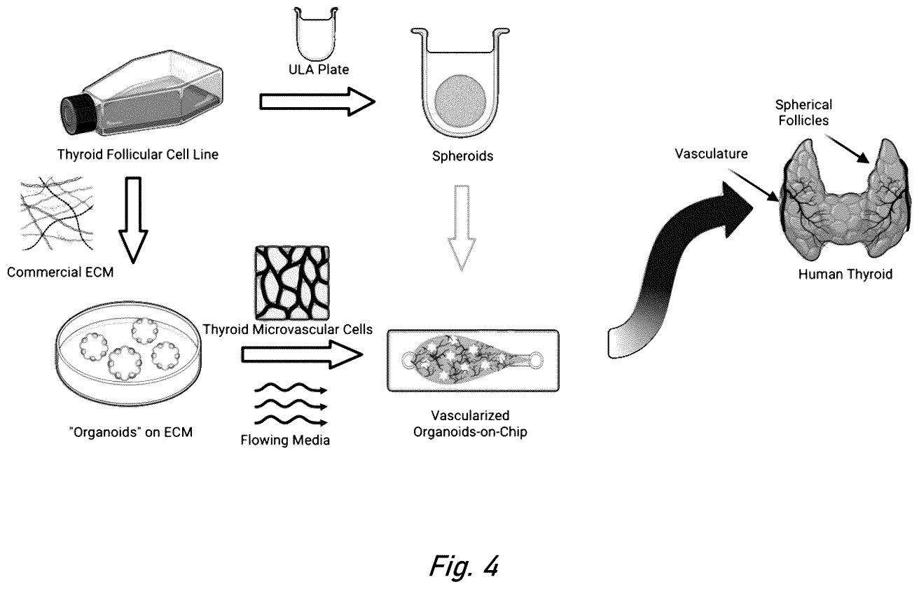

An effective in-vitro model of the thyroid should mimic actual in vivo thyroid by having both thyroid gland cells and thyroid vasculature cells for reliable applications in studying thyroid-immune interactions and predicting patient responses. (See FIGS. 2, 4.)

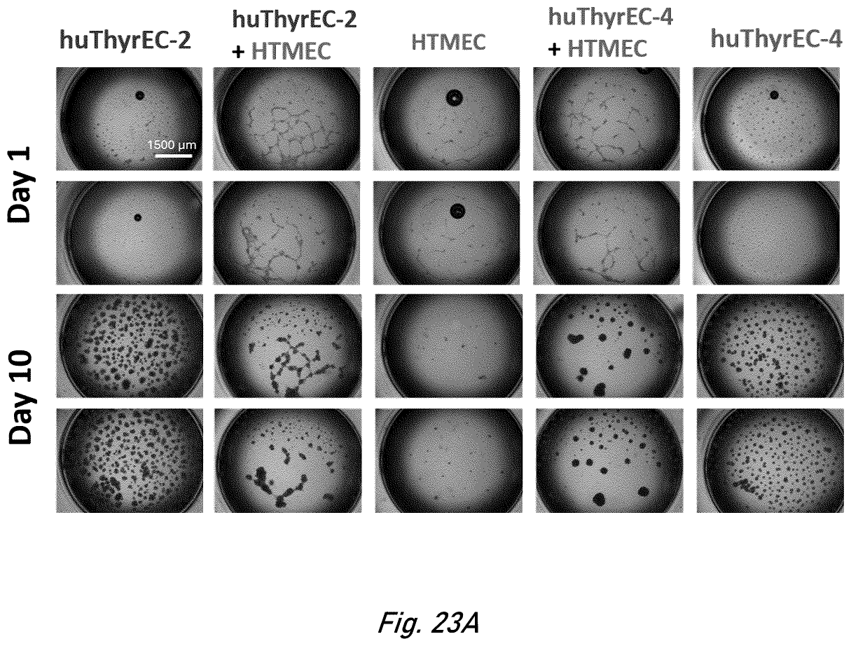

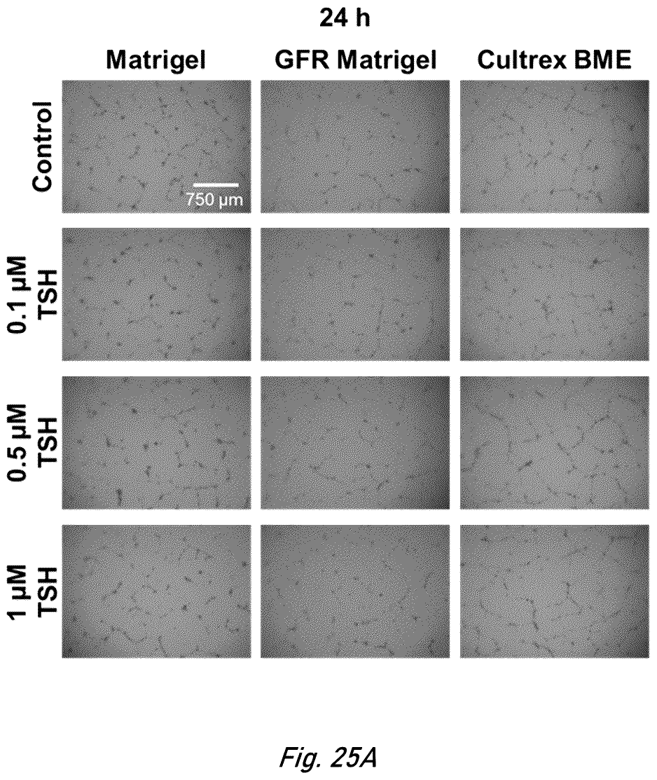

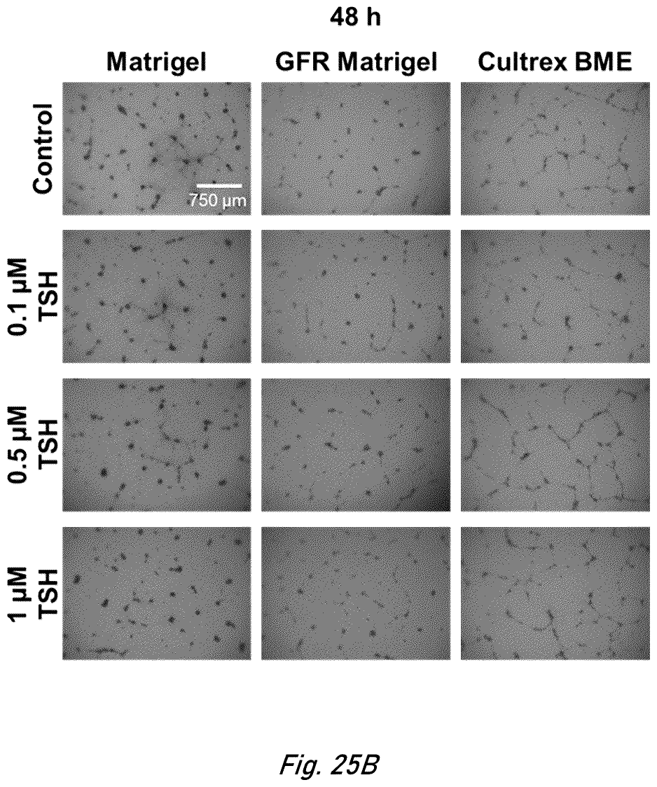

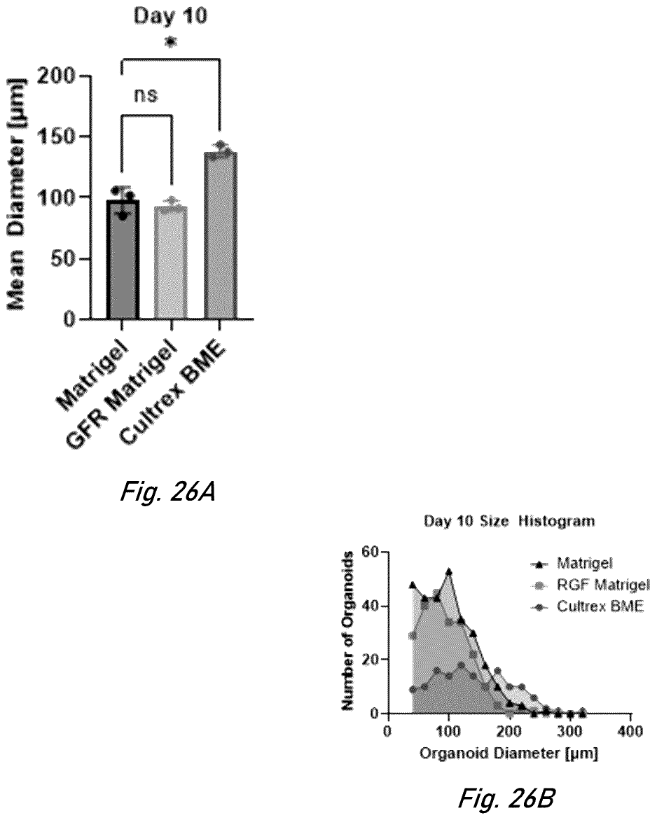

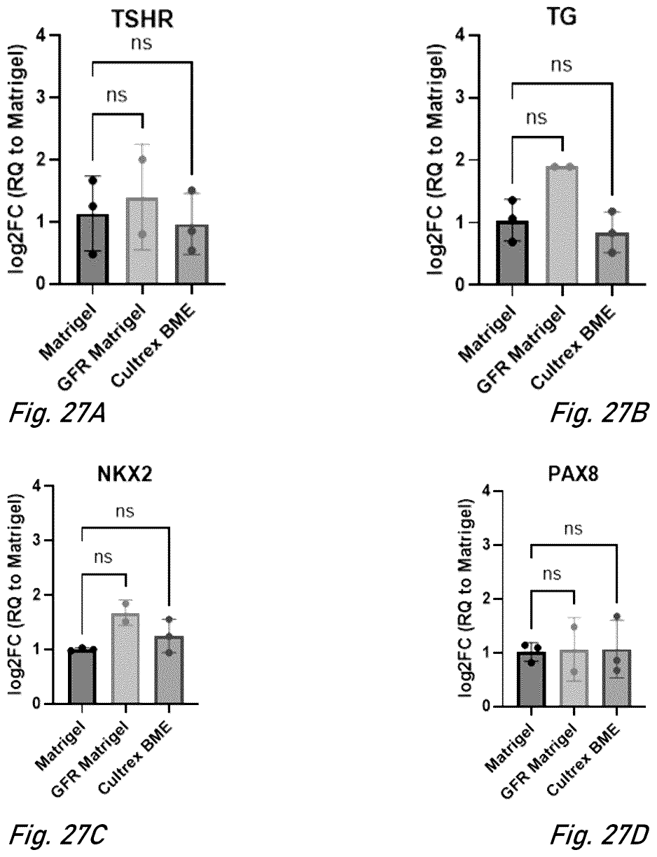

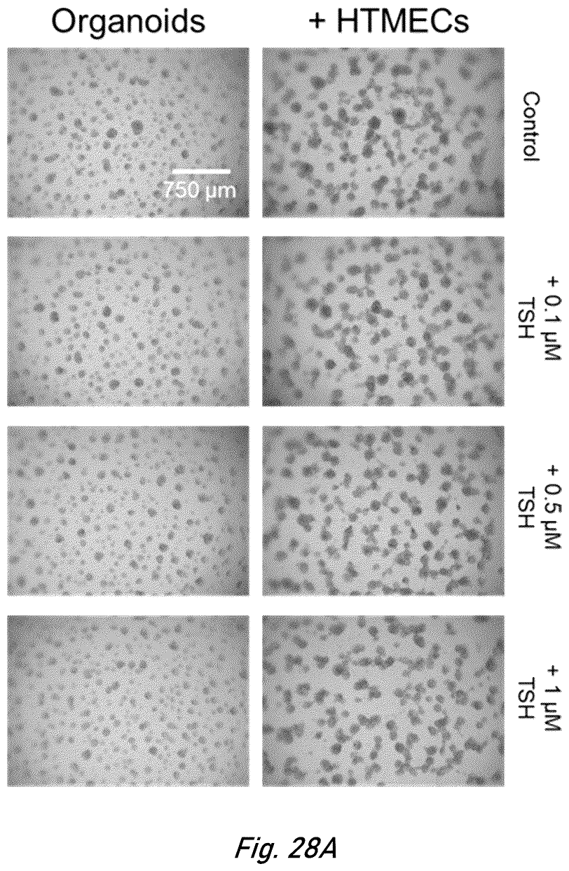

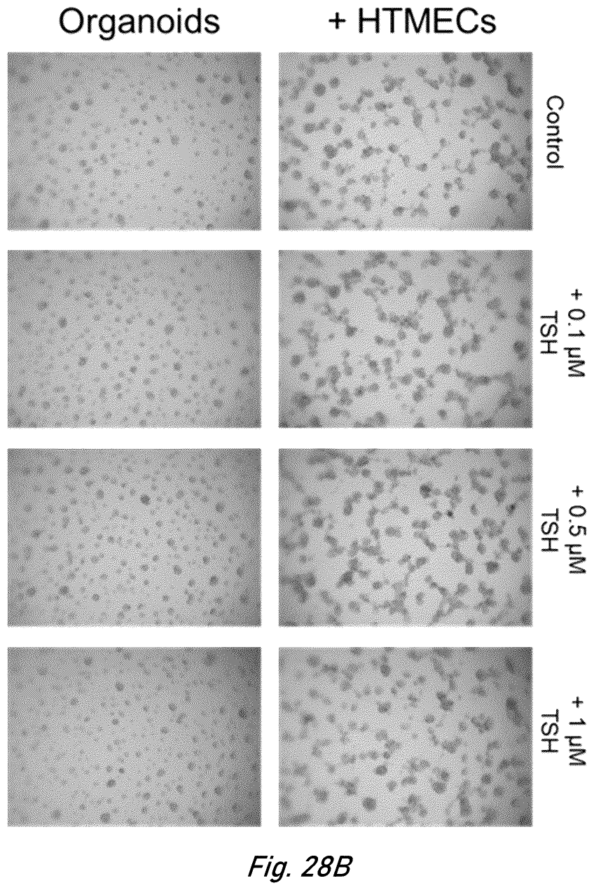

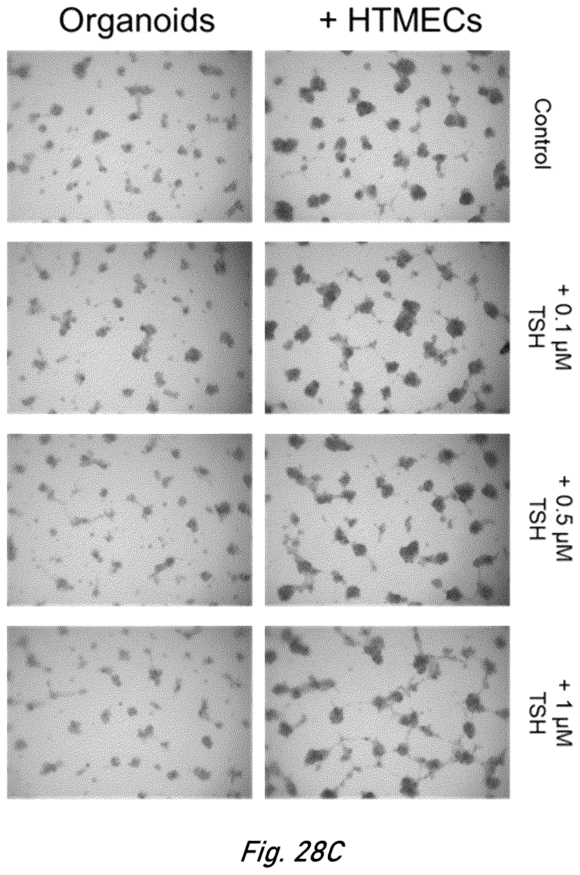

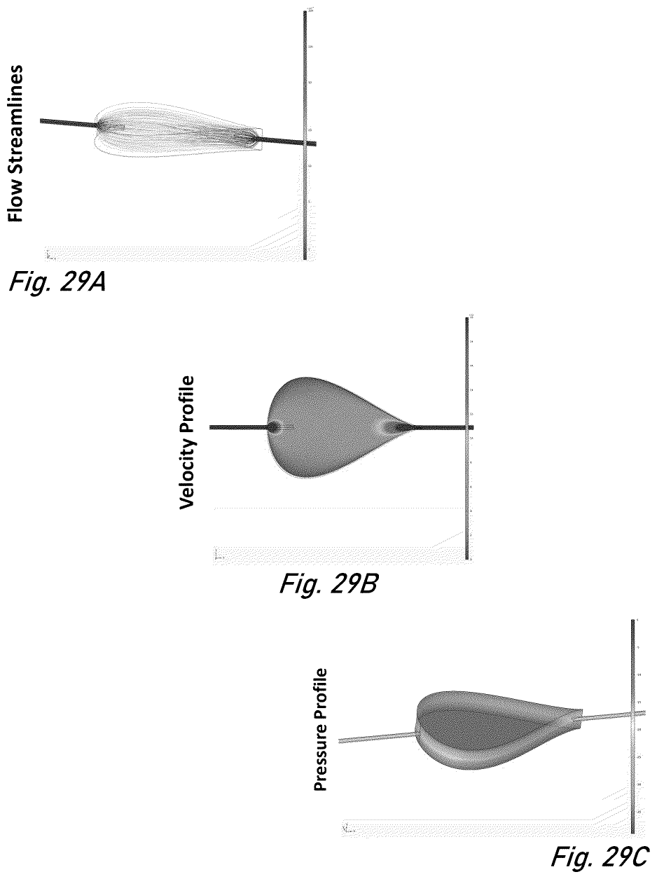

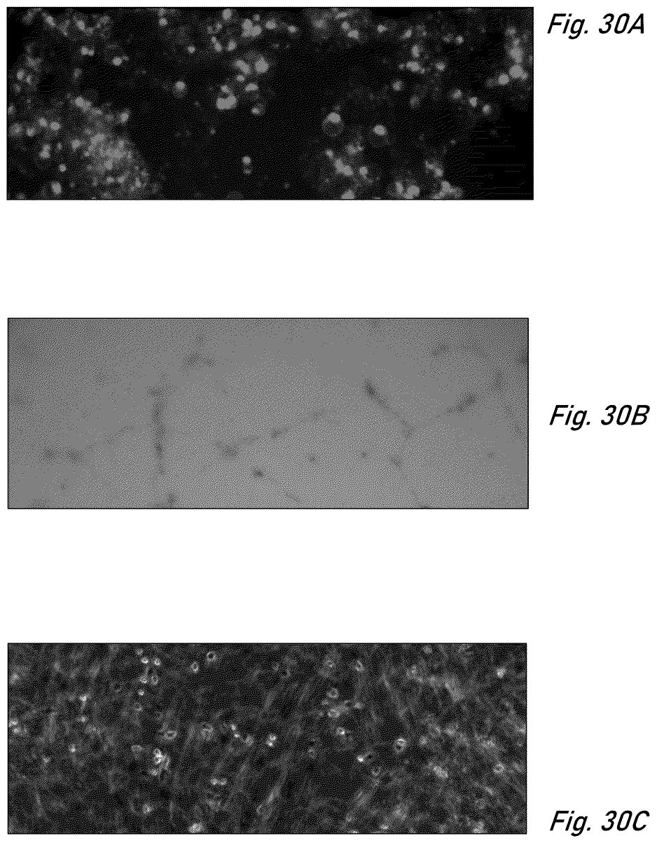

Advantageously, the model of the present disclosure provides a vascularized model of a thyroid. Thus, the present disclosure provides, in one aspect, an an in-vitro model of a thyroid. The present disclosure demonstrates successful monoculturing of primary human thyroid microvascular endothelial cells (HTMECs) on collagen-based gels (See FIGS. 23A-23B, 25A-25B). The present disclosure further demonstrates the successful formation of thyroid follicle cell spheroids, and thyroid follicle-like organoids from immortalized human thyroid epithelial cells (huThyrECs). The present disclosure further demonstrates co-cultures of HTMECs and huThyrECs resulting in the formation of vascularized networks of thyroid-follicle-like organoids (See, e.g., FIGS. 25A-25B, 28A-28C). Data on comparisons between co-cultures on different collagen-based gels validate growth protocols for these co-cultures. (See, e.g., FIGS. 26A-26B, 27A-27D). The results demonstrate a vascularized tissue model with heterogeneous co-culture of thyroid endothelial and thyroid follicular cells. Further experiments demonstrate a vascularized tissue model established by co-culturing of thyroid endothelial cells with follicular organoids within microfluidic devices (FIGS. 29A-29C, 30A-30C). The microfluidic device of the present disclosure has applications in studying healthy and diseased thyroid tissue, as well as testing drugs.

In an aspect, the present disclosure provides an in-vitro model of a thyroid, the model comprising: a co-culture comprising two or more thyroid follicular cell (TFC) spheroids or organoids, and thyroid microvasculature endothelial cell (TMEC) microtubules. In embodiments of the in-vitro model of a thyroid, the co-culture may be a 2D-culture.

In embodiments of the in-vitro model of a thyroid, the co-culture may be a 3D-culture. In embodiments, the 3D co-culture is in an extracellular matrix selected from: standard-formulation reconstituted basement membrane extracted from Engelbreth-Holm-Swarm mouse tumor, growth-factor-reduced reconstituted basement membrane extracted from Engelbreth-Holm-Swarm mouse tumor, soluble basement membrane extracted from Engelbreth-Holm-Swarm mouse tumor, and any combination thereof. In embodiments, the two or more TFC spheroids or organoids are derived from TFC-2 cells, TFC-4 cells, or iPSCs.

As used herein “Matrigel” refers to basement membrane extracted from Engelbreth-Holm-Swarm mouse tumor. As used herein, “spheroid” refers to a collection of cells grown on the surface of an ultra-low-attachment (ULA) well. As used herein, “organoid” refers to a collection of cells embedded in extracellular matrix (ECM).

“HuThyrEC” refers to polyclonal immortalized human thyroid follicular cells. HuThyrEC cell lines, including HuThyrEC-2 and HuThyrEC-4 are derived from a single human male donor of Caucasian decent, 18 years of age, and body mass index of 19. The cell lines were generated by transduction of a lentiviral gene library composed of 33 different genes using methods described in Lipps C. et al. Expansion of functional personalized cells with specific transgene combinations. Nature Communications 9, 994, doi: 10.1038/s41467-018-03408-4 (2018). HuThyrEC-2 and HuThyrEC-4 cells are commercially available from InSCREENex GmbH (InSCREENeX, Braunschweig, Germany).

TMECs as used in the present disclosure refer to primary mammalian TMECs. Human TMECs (HTMECs) may include H-6058 cells, available from Cell Biologics (Cell Biologics, Chicago, IL, USA).

In embodiments of the in-vitro model of a thyroid, the two or more TFC spheroids or organoids are derived from immortalized human thyroid follicular cells. In embodiments, the immortalized human thyroid follicular cells are HuThyrEC cells. In embodiments, the TMEC microtubules are derived from human primary TMECs or iPSCs. In embodiments, the human primary TMECs are H-6058 cells.

“Organ-on-chip” models are microfluidic devices in which tissues can be grown, and which allow researchers to control various conditions in order to mimic the physiological environment that the tissue would be subject to within the body. This may include, e.g., the application of mechanical forces, such as shear stress and flow conditions, as well as the recapitulation of the microenvironment in order to achieve a model with high fidelity. Such devices may be useful to researchers aiming to model various organs due to their improved accuracy in reflecting the in vivo phenotype, as well as their utility in drug testing and the modeling of diseased states.

Despite recent growth in the field, there remain many organs which have yet to be successfully modeled in organ-on-chip models. The thyroid gland is one such organ. An organ-on-a-chip model of the thyroid would provide an opportunity to study the thyroid and its function with greater accuracy and may provide a bridge to the development of personalized medicine models.

The present disclosure provides a vascularized organ-on-a-chip model in order to model thyroid autoimmune diseases and cancer. Thus, in an aspect, the present disclosure provides a thyroid-on-a-chip comprising a microfluidic device having a chamber defining an enclosed volume, having disposed within the volume the in-vitro model of the present disclosure.

In another aspect, the present disclosure provides a method of manufacturing an in-vitro model of a thyroid, the method comprising: co-culturing two or more TFC spheroids or organoids and two or more TMEC microtubules. In embodiments, co-culturing may be 2D co-culturing. In embodiments, the co-culturing may be 3D co-culturing. In embodiments, the 3D co-culturing may be in an extracellular matrix selected from: standard-formulation reconstituted basement membrane extracted from Engelbreth-Holm-Swarm mouse tumor, growth-factor-reduced reconstituted basement membrane extracted from Engelbreth-Holm-Swarm mouse tumor, soluble basement membrane extracted from Engelbreth-Holm-Swarm mouse tumor, and any combination thereof. In embodiments, the two or more TFC spheroids or organoids may be derived from TFC-2 cells, TFC-4 cells, or induced pluripotent stem cells (iPSCs). In embodiments, the two or more TFC spheroids or organoids may be derived from immortalized human thyroid follicular cells. In embodiments, the immortalized human thyroid follicular cells may be HuThyrEC cells. In embodiments, the TMEC microtubules may be derived from human primary TMECs or iPSCs. In embodiments, the human primary TMECs may be H-6058 cells.

In another aspect, the present disclosure provides a microfluidic thyroid model device comprising: a microfluidic chamber having disposed therein a co-culture comprising thyroid follicular cells and thyroid microvasculature endothelial cells. In embodiments of the microfluidic thyroid model device, the device comprises a gel disposed within the chamber, wherein at least a portion of the co-culture is disposed within the gel. In embodiments, the gel comprises an extracellular matrix (ECM), wherein the ECM is selected from: standard-formulation reconstituted basement membrane extracted from Engelbreth-Holm-Swarm mouse tumor, growth-factor-reduced reconstituted basement membrane extracted from Engelbreth-Holm-Swarm mouse tumor, soluble basement membrane extracted from Engelbreth-Holm-Swarm mouse tumor, and any combination thereof. In embodiments, the thyroid follicular cells are HuThyrEC cells. In embodiments, the thyroid microvasculature endothelial cells are H-6058 primary human TMECs.

In embodiments of the microfluidic thyroid model device, the microfluidic chamber is tapered, wherein the teardrop shape defines a wide portion and a narrow portion, and wherein the wide portion of the tapered microfluidic chamber is in fluidic communication with a fluid inlet, and the narrow portion of the tapered microfluidic chamber is in fluidic communication with a fluid outlet. By tapering the shape of the chamber, having a wider portion near the inlet and narrower portion near the outlet, a fluid flow profile and pressure gradient may be generated inside the chamber in a way which mimics the progressive narrowing of vasculature in vivo.

In embodiments of the microfluidic thyroid model device, the microfluidic chamber may be teardrop shaped, wherein the teardrop shape defines a wide portion and a narrow tapered portion, and wherein the wide portion of the teardrop-shaped microfluidic chamber is in fluidic communication with a fluid inlet, and the narrow tapered portion of the teardrop-shaped microfluidic chamber is in fluidic communication with a fluid outlet. Persons skilled in the art will recognize that many alternative shapes may be used.

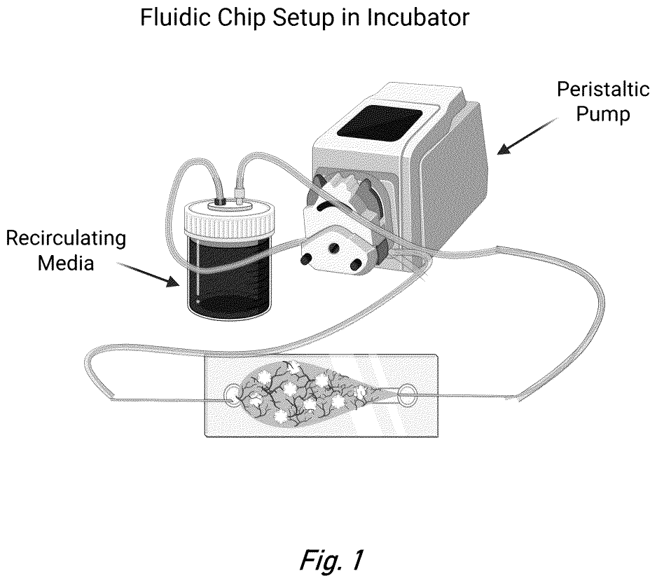

Some embodiments of the microfluidic thyroid model device further comprise a pump which causes fluid medium to flow into to fluid inlet, through the chamber, and out the fluid outlet, such that the fluid medium contacts the co-culture as it flows through the chamber. In embodiments, the fluid medium comprises one or more test agents. In embodiments, the one or more test agents is selected from: a thyroid autoimmunity test agent or a thyroid cancer therapeutic agent. (See FIG. 1.)

In another aspect, the present disclosure provides a method of testing an agent, the method comprising: (a) contacting the agent with the in-vitro model of the present disclosure, wherein the agent contacting the in-vitro model is suspended in a fluid which is in continuous flow across or through the in-vitro model, and wherein the fluid imparts a shear force on the cells of the in-vitro model; and (b) analyzing the in-vitro model for a response to the agent. Analysis may be performed by techniques known in the art including, e.g., PCR, qPCR, RT-PCR, immunofluorescence staining, or Western blot. The shear forces and fluid pressure imparted on the co-culture model mimic in vivo conditions, providing a higher fidelity, reliable in vitro model. In any embodiment, the test agent may comprise an experimental reagent. In any embodiment, the test agent may comprise a therapeutic agent and/or candidate therapeutic agent. In embodiments, the shear force the fluid imparts on the cells of the in-vitro model is about the same as the shear force that blood serum imparts on thyroid follicular cells in vivo.

In yet another aspect, the present disclosure provides a thyroid-on-a-chip comprising: a microfluidic device comprising a microfluidic chamber in fluidic communication with a fluid inlet and a fluid outlet; wherein, disposed within the microfluidic chamber, is a vascularized thyroid organoid co-culture comprising two or more thyroid follicular cell (TFC) spheroids or organoids in 3D-culture with thyroid microvasculature endothelial cell (TMEC) microtubules, wherein the TFC spheroids comprise HuThyrEC cells and wherein the TMEC microtubules comprise H-6058 cells, wherein the 3D-culture is in an extracellular matrix selected from: standard-formulation reconstituted basement membrane extracted from Engelbreth-Holm-Swarm mouse tumor, growth-factor-reduced reconstituted basement membrane extracted from Engelbreth-Holm-Swarm mouse tumor, soluble basement membrane extracted from Engelbreth-Holm-Swarm mouse tumor, and any combination thereof. In embodiments, the thyroid-on-a-chip comprises a a vascularized thyroid organoid co-culture comprising two or more TFC spheroids in 3D-culture with TMEC microtubules. In embodiments, the thyroid-on-a-chip comprises a a vascularized thyroid organoid co-culture comprising two or more TFC organoids in 3D-culture with TMEC microtubules.

### EXAMPLES

The following examples further illustrate contemplated products and methods of the present disclosure, but should not be construed as in any way limiting its scope.

### Example 1. Co-Culture of Human Thyroid Follicular Cells and Human Thyroid Microvascular Endothelial Cells

Human thyroid microvascular endothelial cells (HTMECs) and human thyroid epithelial cells from two cell lines-huThyrEC-2 (FIG. 23A) and huThyrEC-4 (FIG. 23B)-were seeded onto Matrigel (left of both FIGS. 23A and 23B) and Cultrex Basement Membrane Extract (right of both FIGS. 23A and 23B). Monoculture of cells was compared to co-culture. HuThyrEC-2 and huThyrEC-4 cells formed organoids in monoculture. HTMEC monoculture formed delicate, tubular networks. These networks were maintained on BME throughout the course of the experiment. Co-culture showed network formation, without apparent separation between the two cell lines.

### Example 2. Thyroid Follicular Cell Spheroid Formation

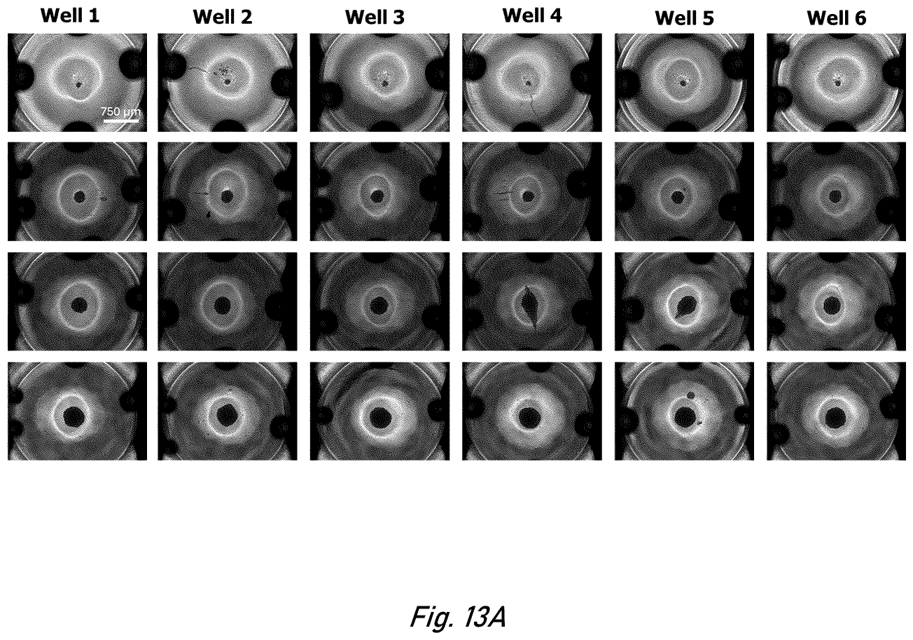

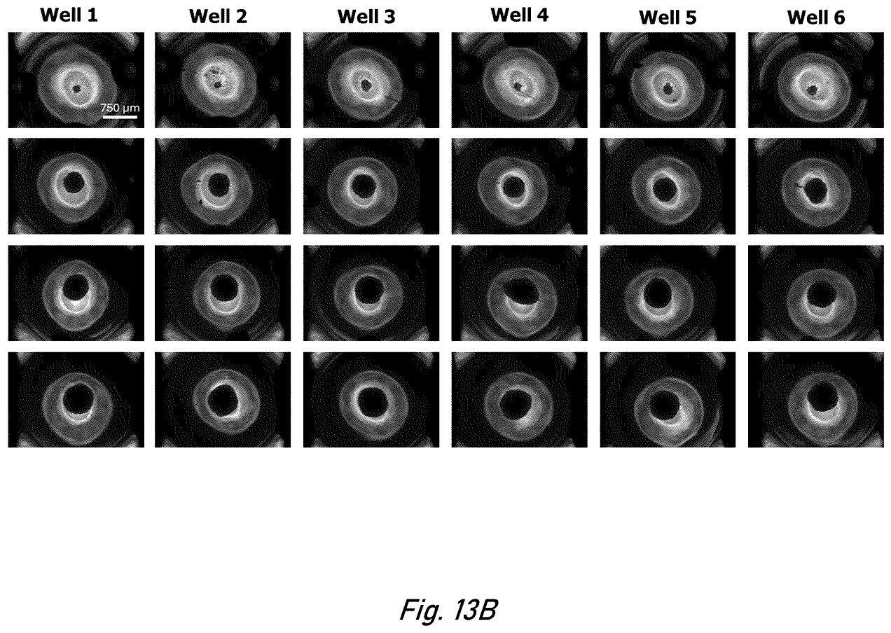

Spheroids were formed in a 384-well plate. Imaging at day 3 (FIG. 13A) and day 5 (FIG. 13B) after plating shows that increased seeding density resulted in larger spheroids, as did longer time in culture. The images in FIGS. 13A-13B show huThyrEC-4 spheroids.

### Example 3. Organoids by Varying Seeding Density

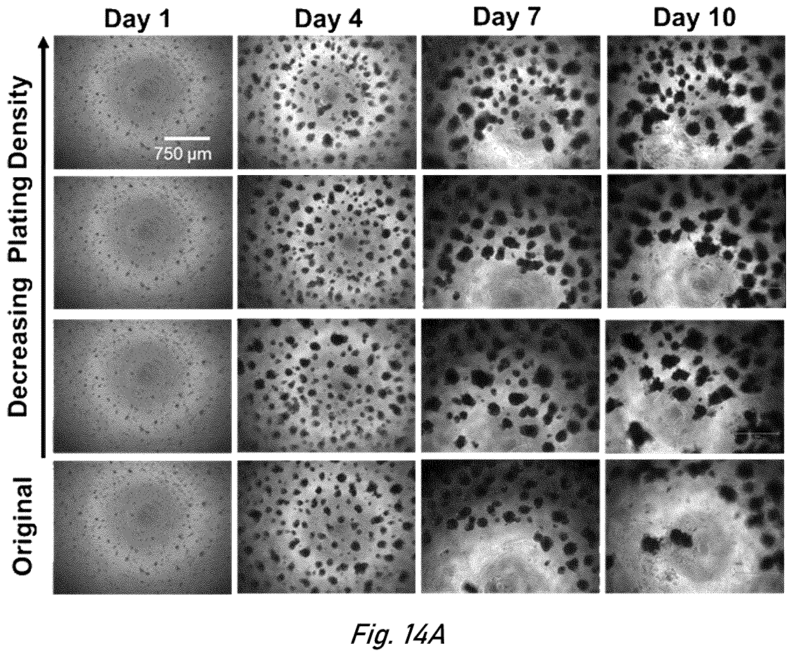

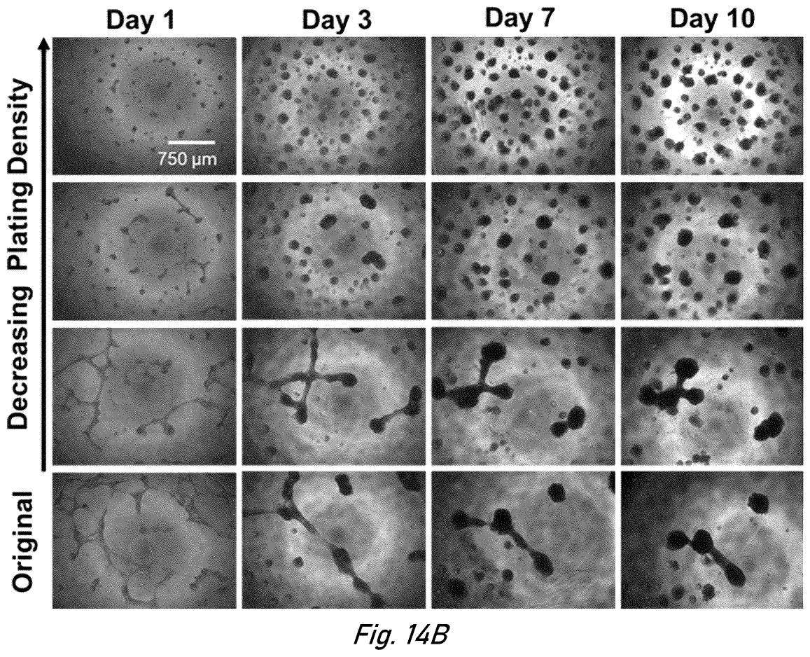

Thyroid organoids were formed by HuThyrEC-2 cells (FIG. 14A) and huThyrEC-4 cells (FIG. 14B) seeded onto Matrigel at various seeding densities, and organoid formation was compared. Lower seeding densities and less time in culture yielded organoids with less network formation.

### Example 4. Validation of Immortalized Human Thyroid Follicular Cells

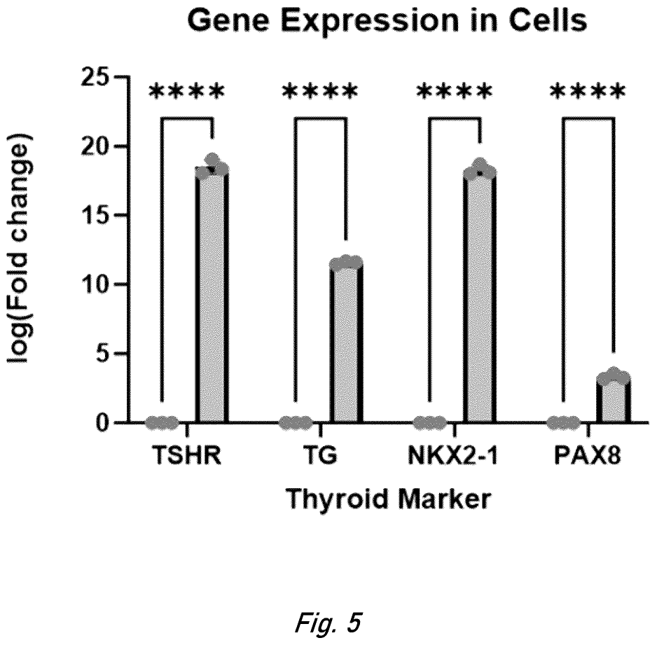

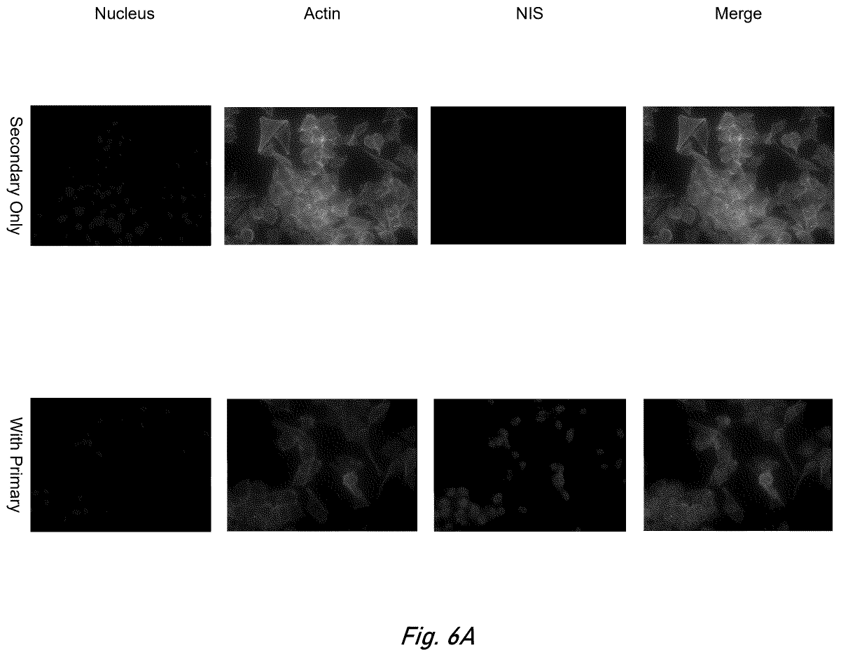

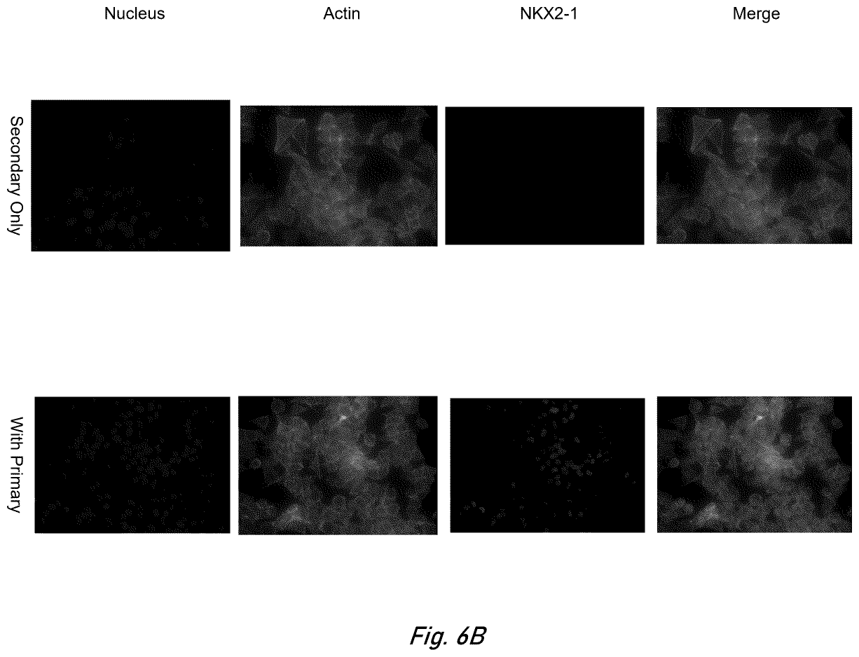

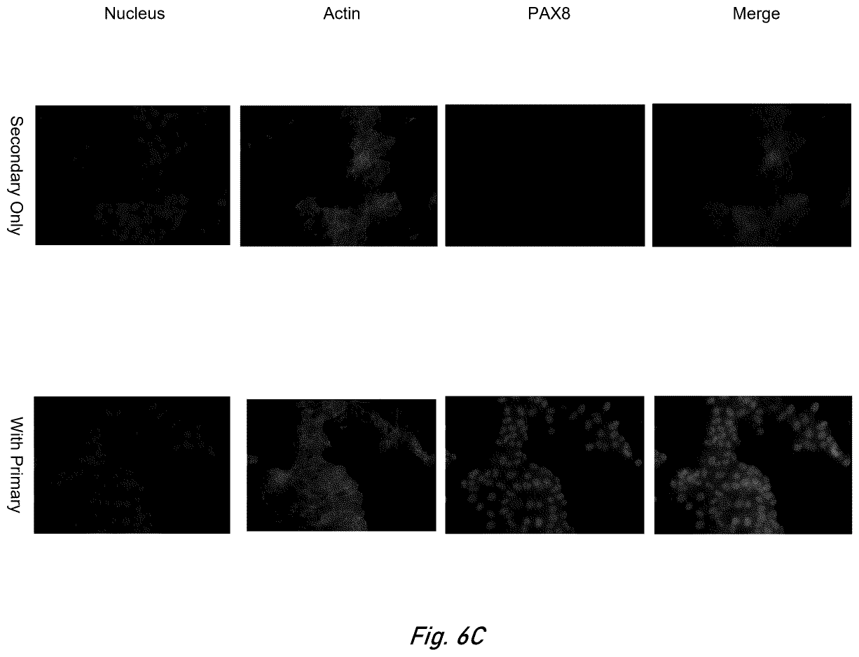

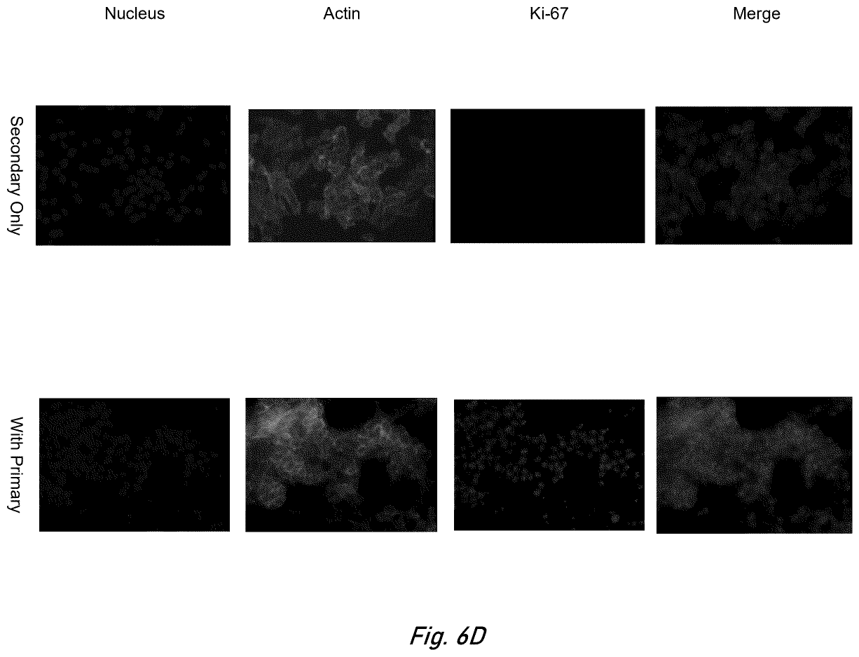

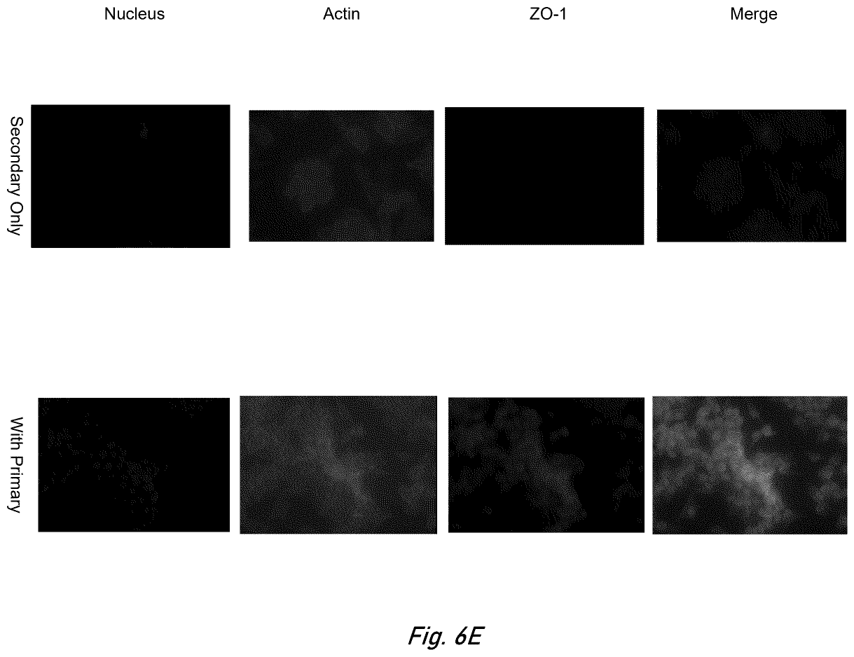

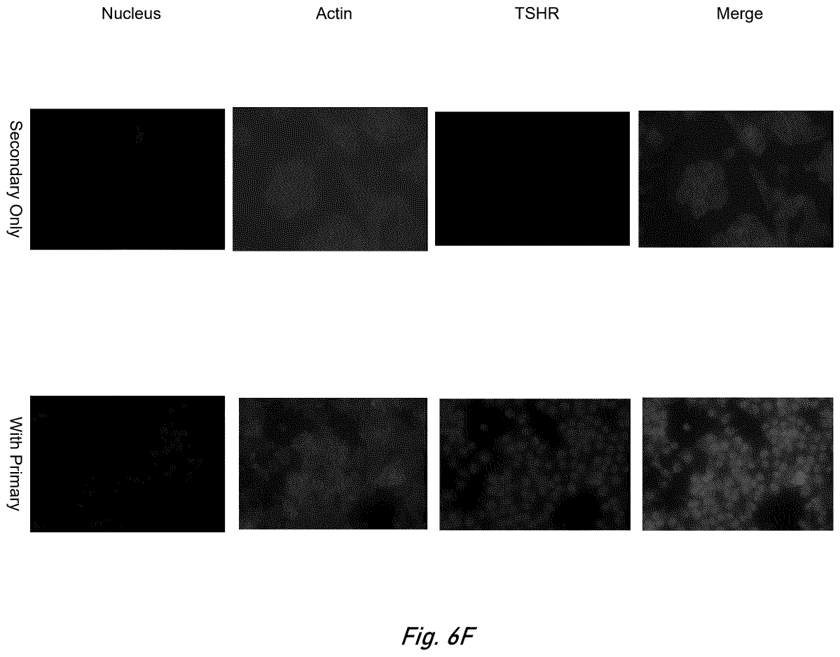

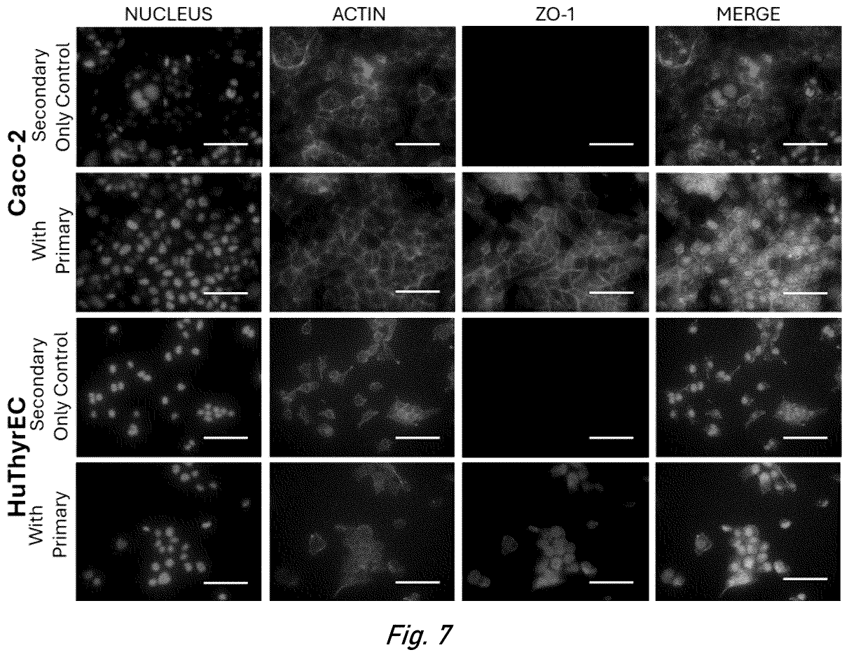

To ensure that HuThyrEC cells provide a reliable and accurate replicate of in vivo thyroid, HuThyrEC cells and HuThyrEC spheroids were tested for various thyroid cell markers by RT-PCR (FIG. 5) and fluorescence microscopy (FIGS. 6A-6F). The results show the HuThyrEC cells of the present disclosure have the expression profile of in vivo thyroid. FIG. 7 depicts fluorescence microscopy results confirmatory experiments comparing ZO-1 expression of huThyrEC cells and intestinal epithelium Caco-2 cells (positive control for ZO-1).

### Example 5. Growth of Spheroids From Immortalized Human Thyroid Follicular Cells

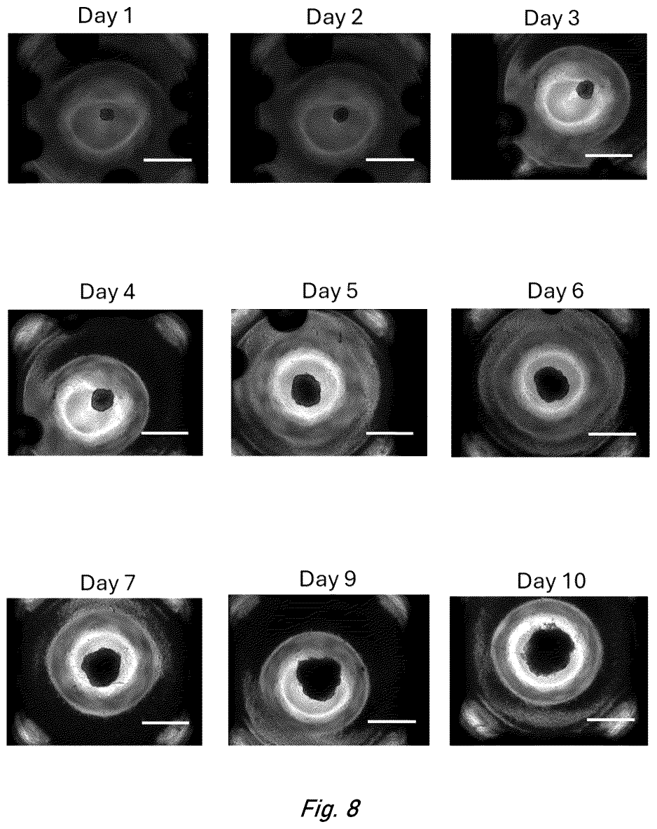

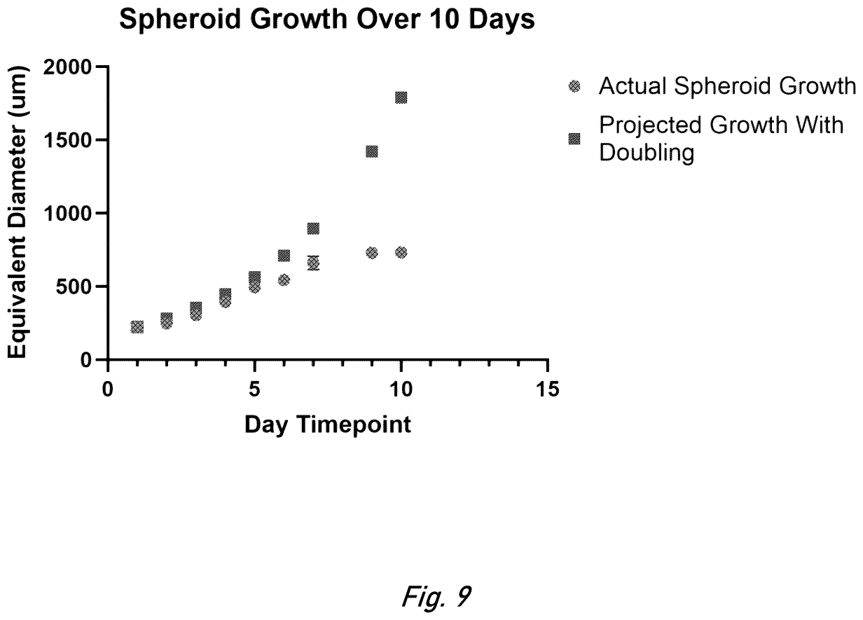

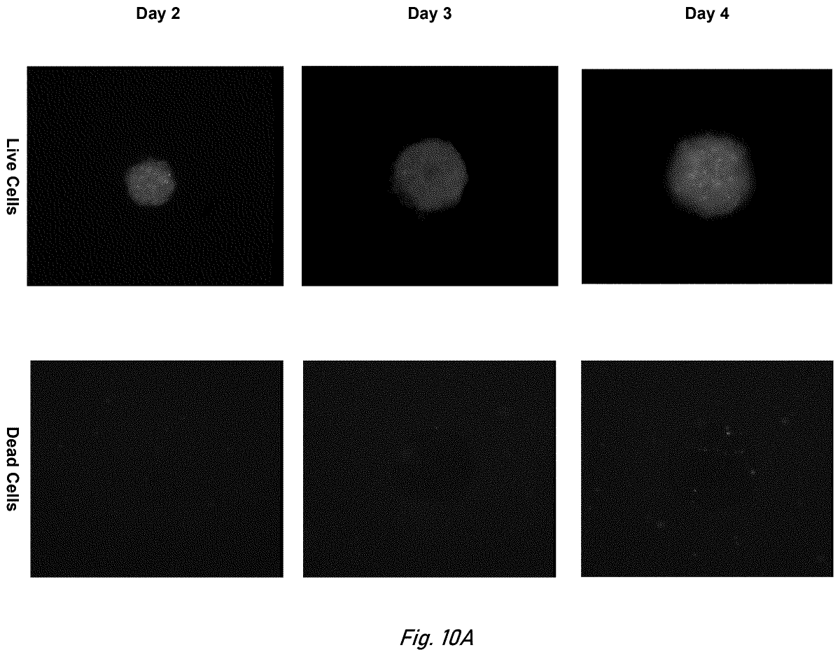

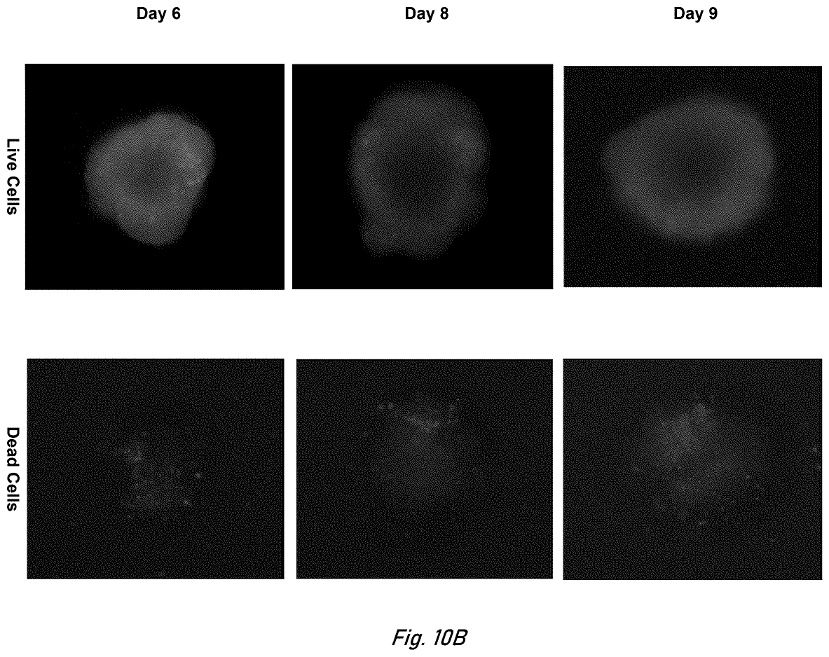

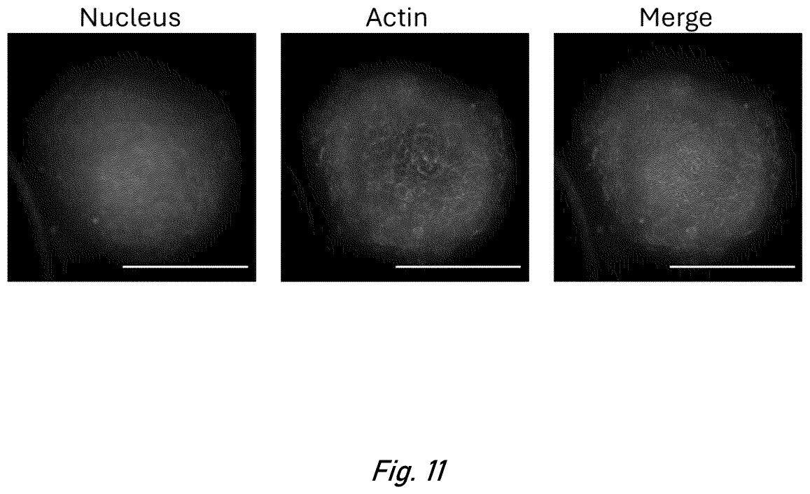

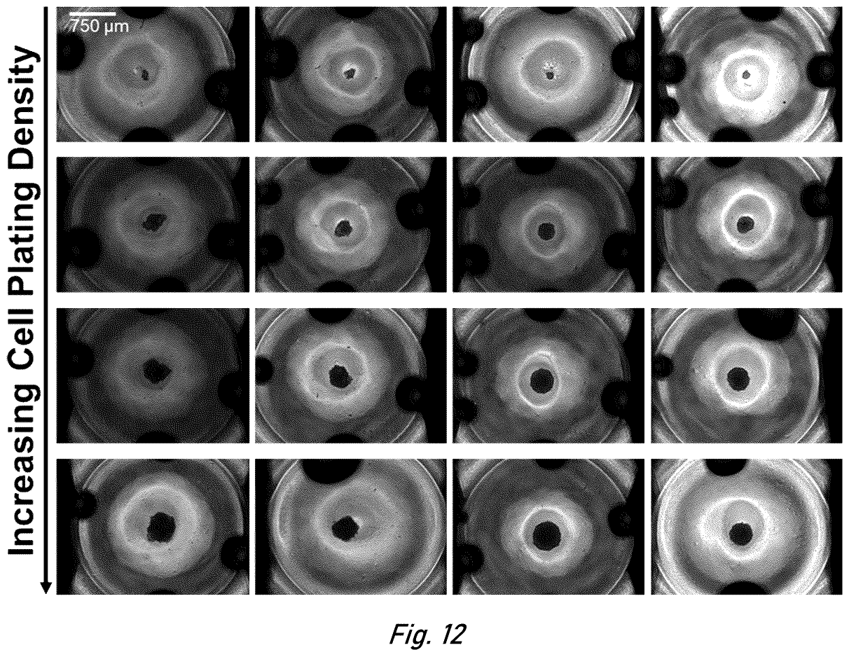

HuThyrEC were seeded in ultra-low attachment wells and characterized after a ten day growth timecourse. Results of the experiment are shown in FIGS. 8, 9, 10A-10B, 11, 12, and 13A-13B.

### Example 6. Characterization of Organoids Grown and From Immortalized Human Thyroid Follicular Cells

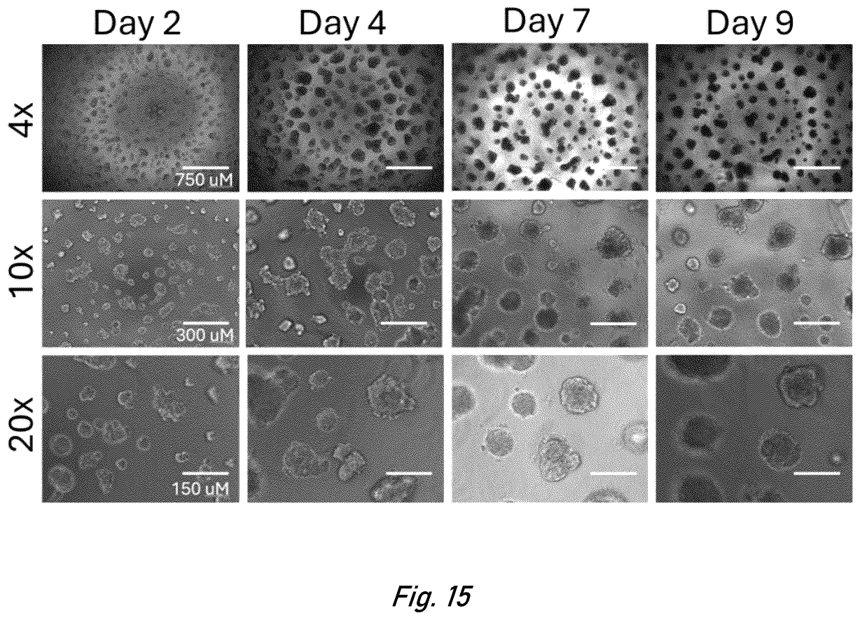

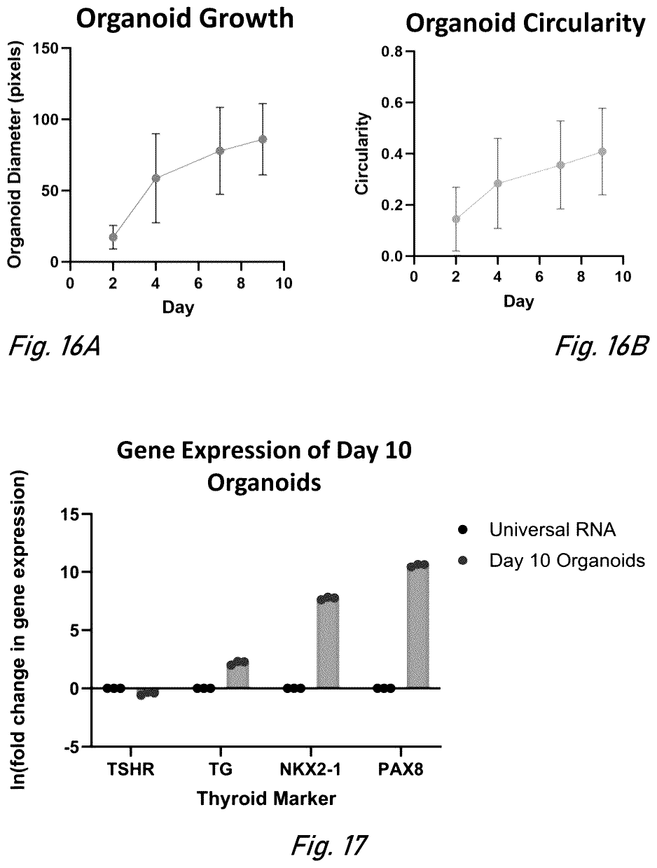

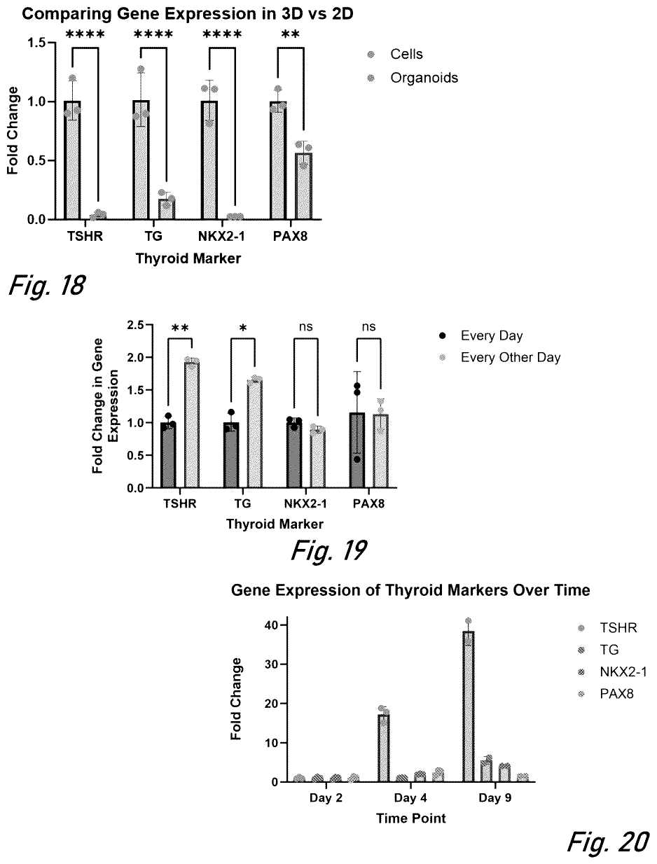

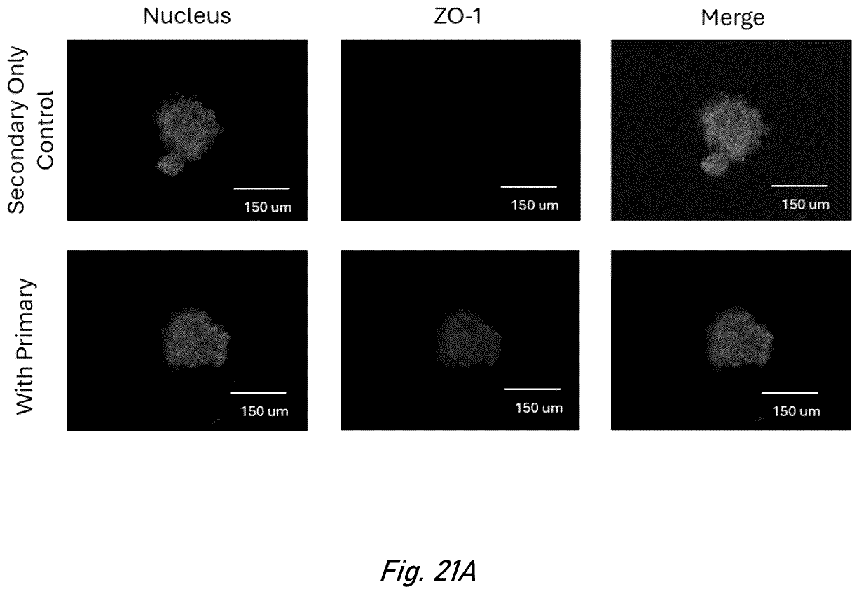

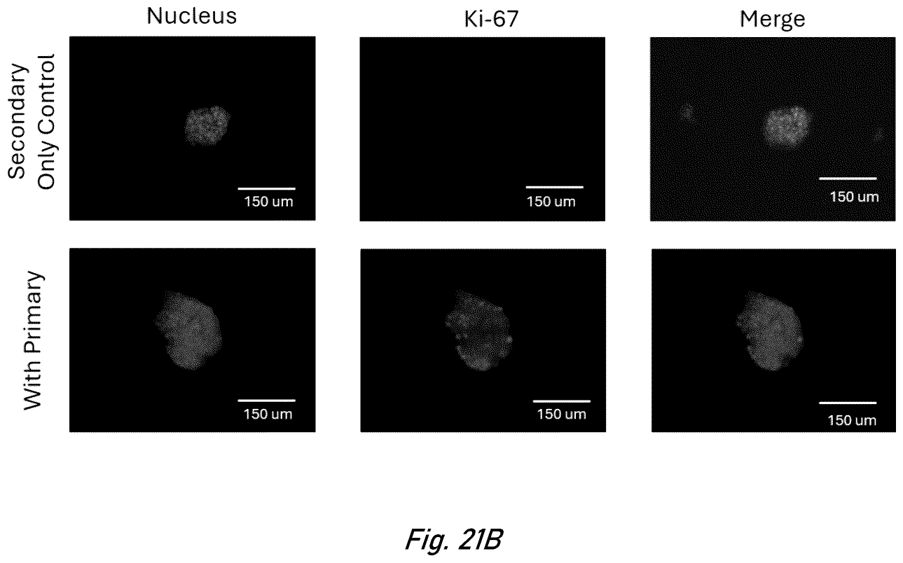

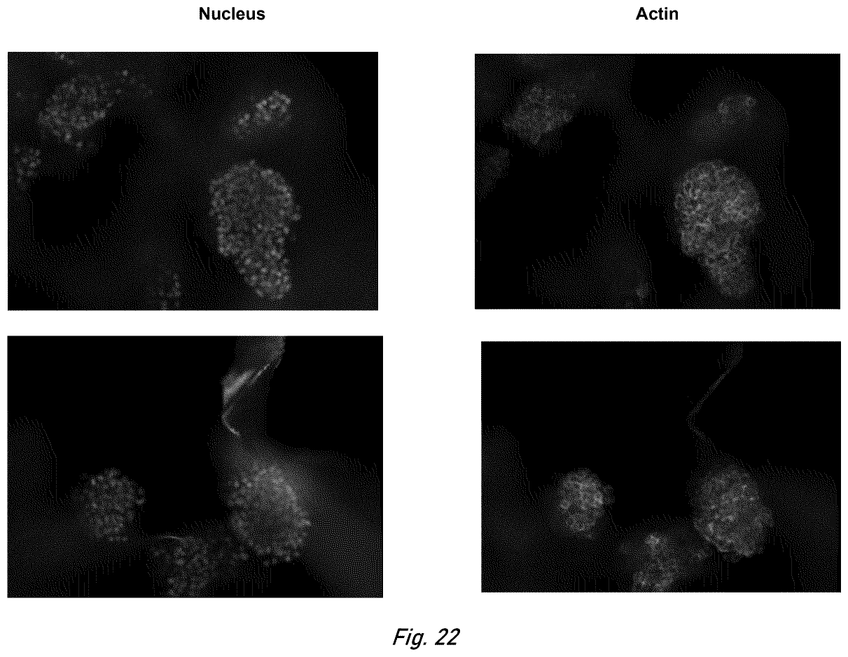

HuThyrEC were seeded embedded in ECM and characterized after a ten day growth timecourse. Results of the experiment are shown in FIGS. 14A-14B, 15, 16A-16B, 17, 18, 19, 20, 21A-21B, and 22.

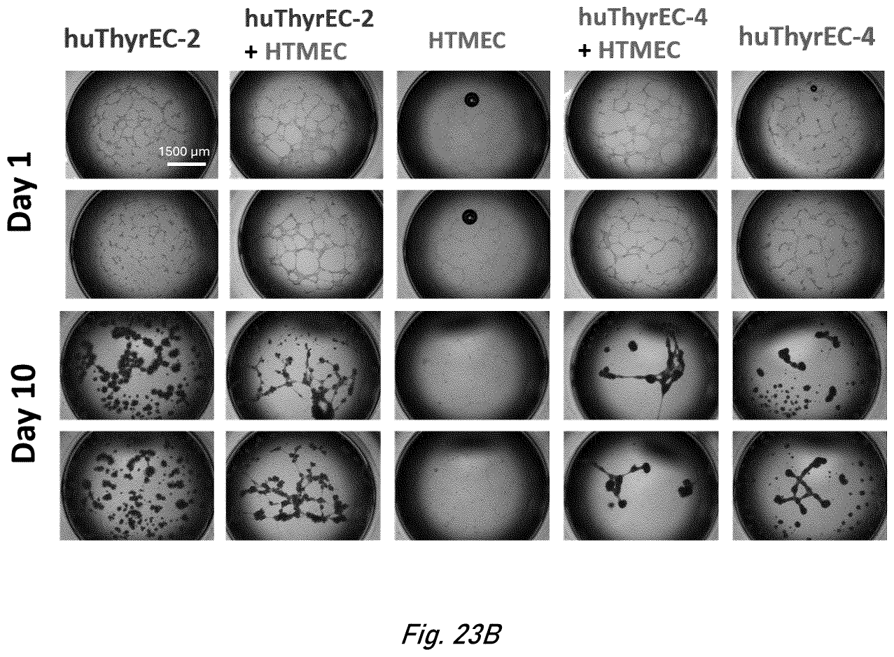

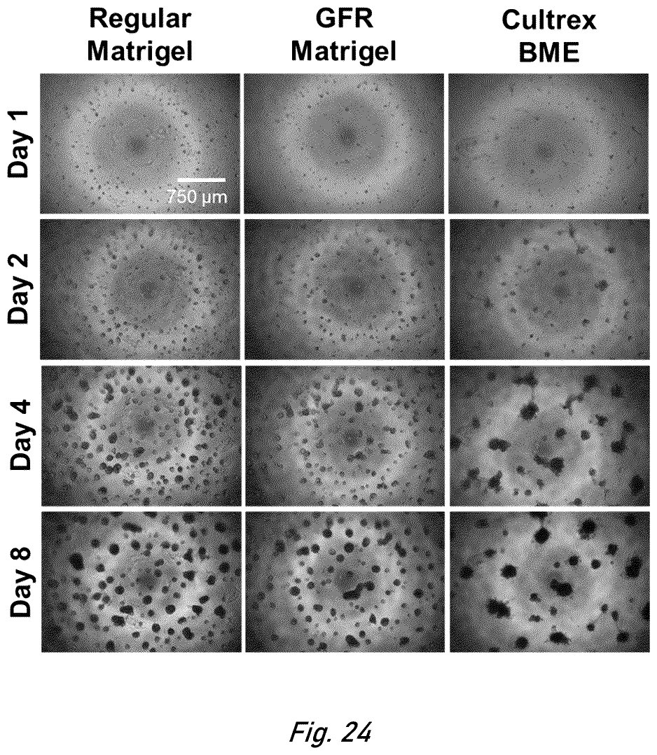

Organoids were tested in three different 3D culture growth media: Matrigel®; growth-factor-reduced (GFR) Matrigel®; and Cultrex™ BME. (See FIG. 24.) Dimensional analysis performed on digital images found mean diameter of organoids was significantly larger in Cultrex™ BME. (See FIGS. 26A-26B.)

### Example 7. Modeling of Microfluidics

A microfluidic device was modeled using computer simulation (FIGS. 29A-29C). A tear-drop shaped chamber was selected because the fluid flow and pressure gradient is highly similar to that of blood flowing in vasculature of in vivo thyroid from larger vessels to small vessels (e.g., from arteries to arterioles to capillaries). Co-culture was embedded in a chip, and modeled with a peristaltic pump pushing fluid through the chip. (Co-cultured cells in the chip shown in FIGS. 30A-30C.)

The terms “about” and “around,” as used herein to modify a numerical value, indicate a close range surrounding the numerical value. Thus, if “X” is the value, “about X” or “around X” indicates a value of from 0.9X to 1.1X, e.g., from 0.95X to 1.05X or from 0.99X to 1.01X. A reference to “about X” or “around X” specifically indicates at least the values X, 0.95X, 0.96X, 0.97X, 0.98X, 0.99X, 1.01X, 1.02X, 1.03X, 1.04X, and 1.05X. Accordingly, “about X” and “around X” are intended to teach and provide written description support for a claim limitation of, e.g., “0.98X.”

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and “at least one” and similar referents in the context of describing the invention (especially in the context of the following claims) are to be construed to cover both the singular and the plural, unless otherwise indicated herein or clearly contradicted by context. The use of the term “at least one” followed by a list of one or more items (for example, “at least one of A and B”) is to be construed to mean one item selected from the listed items (A or B) or any combination of two or more of the listed items (A and B), unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. Skilled artisans may be expected to employ such variations as appropriate, and the invention may be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. An in-vitro model of a thyroid, the model comprising: a co-culture comprising two or more thyroid follicular cell (TFC) spheroids or organoids, and thyroid microvasculature endothelial cell (TMEC) microtubules.

2. The in-vitro model of claim 1, wherein the co-culture is a 3D co-culture.

3. The in-vitro model of claim 2, wherein the 3D co-culture is in an extracellular matrix selected from: standard-formulation reconstituted basement membrane extracted from Engelbreth-Holm-Swarm mouse tumor, growth-factor-reduced reconstituted basement membrane extracted from Engelbreth-Holm-Swarm mouse tumor, soluble basement membrane extracted from Engelbreth-Holm-Swarm mouse tumor, and any combination thereof.

4. The in-vitro model of claim 1, wherein the two or more TFC spheroids or organoids are derived from TFC-2 cells, TFC-4 cells, or iPSCs.

5. The in-vitro model of claim 1, wherein the two or more TFC spheroids or organoids are derived from immortalized human thyroid follicular cells.

6. The in-vitro model of claim 5, wherein the immortalized human thyroid follicular cells are HuThyrEC cells.

7. The in-vitro model of claim 1, wherein the TMEC microtubules are derived from human primary TMECs or iPSCs.

8. The in-vitro model of claim 7, wherein the human primary TMECs are H-6058 cells.

9. A thyroid-on-a-chip comprising a microfluidic device having a chamber defining an enclosed volume, having disposed within the volume the in-vitro model of claim 1.

10. A microfluidic thyroid model device comprising: a microfluidic chamber having disposed therein a co-culture comprising thyroid follicular cells and thyroid microvasculature endothelial cells.

11. The microfluidic thyroid model device of claim 10, wherein the device comprises a gel disposed within the chamber, wherein at least a portion of the co-culture is disposed within the gel.

12. The microfluidic thyroid model device of claim 11, wherein the gel comprises an extracellular matrix (ECM), wherein the ECM is selected from: standard-formulation reconstituted basement membrane extracted from Engelbreth-Holm-Swarm mouse tumor, growth-factor-reduced reconstituted basement membrane extracted from Engelbreth-Holm-Swarm mouse tumor, soluble basement membrane extracted from Engelbreth-Holm-Swarm mouse tumor, and any combination thereof.

13. The microfluidic thyroid model device of claim 10, wherein the thyroid follicular cells are HuThyrEC cells.

14. The microfluidic thyroid model device of claim 10, wherein the thyroid microvasculature endothelial cells are H-6058 primary human TMECs.

15. The microfluidic thyroid model device of claim 10, wherein the microfluidic chamber is teardrop shaped,
wherein the teardrop shape defines a wide portion and a narrow tapered portion, and wherein the wide portion of the teardrop-shaped microfluidic chamber is in fluidic communication with a fluid inlet, and the narrow tapered portion of the teardrop-shaped microfluidic chamber is in fluidic communication with a fluid outlet.

16. The microfluidic thyroid model device of claim 15, further comprising a pump which causes fluid medium to flow into to fluid inlet, through the chamber, and out the fluid outlet, such that the fluid medium contacts the co-culture as it flows through the chamber.

17. The microfluidic thyroid model device of claim 16, wherein the fluid medium comprises one or more test agents.

18. The microfluidic thyroid model device of claim 17, wherein the one or more test agents is selected from: a thyroid autoimmunity test agent or a thyroid cancer therapeutic agent.

19. A method of testing an agent, the method comprising:
(a) contacting the agent with the in-vitro model of claim 1, wherein the agent contacting the in-vitro model is suspended in a fluid which is in continuous flow across or through the in-vitro model, and wherein the fluid imparts a shear force on the cells of the in-vitro model; and
(b) analyzing the in-vitro model for a response to the agent.

20. A thyroid-on-a-chip comprising:
a microfluidic device comprising a microfluidic chamber in fluidic communication with a fluid inlet and a fluid outlet;
wherein, disposed within the microfluidic chamber, is a vascularized thyroid organoid co-culture comprising two or more thyroid follicular cell (TFC) spheroids in 3D-culture with thyroid microvasculature endothelial cell (TMEC) microtubules,
wherein the TFC spheroids comprise HuThyrEC cells and wherein the TMEC microtubules comprise H-6058 cells,
wherein the 3D-culture is in an extracellular matrix selected from: standard-formulation reconstituted basement membrane extracted from Engelbreth-Holm-Swarm mouse tumor, growth-factor-reduced reconstituted basement membrane extracted from Engelbreth-Holm-Swarm mouse tumor, soluble basement membrane extracted from Engelbreth-Holm-Swarm mouse tumor, and any combination thereof.
